SEQUENCE LISTING (50 sequences)

SEQ ID NO: 1 - protein, 5 aa, synthetic construct
YSWIN

SEQ ID NO: 2 - protein, 17 aa, synthetic construct
RIFPGDGDTDYNGKFKG

SEQ ID NO: 3 - protein, 10 aa, synthetic construct
NVFDGYWLVY

SEQ ID NO: 4 - protein, 16 aa, synthetic construct
RSSKSLLHSNGITYLY

SEQ ID NO: 5 - protein, 7 aa, synthetic construct
QMSNLVS

SEQ ID NO: 6 - protein, 9 aa, synthetic construct
AQNLELPYT

SEQ ID NO: 7 - protein, 119 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 8 - protein, 112 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 9 - protein, 5 aa, synthetic construct
TYAMN

SEQ ID NO: 10 - protein, 19 aa, synthetic construct
RIRSKYNNYATYYADSVKG

SEQ ID NO: 11 - protein, 14 aa, synthetic construct
HGNFGNSYVSWFAY

SEQ ID NO: 12 - protein, 14 aa, synthetic construct
GSSTGAVTTSNYAN

SEQ ID NO: 13 - protein, 7 aa, synthetic construct
GTNKRAP

SEQ ID NO: 14 - protein, 9 aa, synthetic construct
ALWYSNLWV

SEQ ID NO: 15 - protein, 125 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 16 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 17 - protein, 672 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGSQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 18 - protein, 447 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 19 - protein, 232 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 219 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 219 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 452 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 213 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 449 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 25 - protein, 219 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 10 aa, synthetic construct
GYTFTSYNMH

SEQ ID NO: 27 - protein, 17 aa, synthetic construct
AIYPGNGDTSYNQKFKG

SEQ ID NO: 28 - protein, 13 aa, synthetic construct
VVYYSNSYWYFDV

SEQ ID NO: 29 - protein, 10 aa, synthetic construct
RASSSVSYMH

SEQ ID NO: 30 - protein, 7 aa, synthetic construct
APSNLAS

SEQ ID NO: 31 - protein, 9 aa, synthetic construct
QQWSFNPPT

SEQ ID NO: 32 - protein, 122 aa, synthetic construct [sequence omitted]

SEQ ID NO: 33 - protein, 106 aa, synthetic construct [sequence omitted]

SEQ ID NO: 34 - protein, 5 aa, synthetic construct
NYYIH

SEQ ID NO: 35 - protein, 17 aa, synthetic construct
WIYPGDGNTKYNEKFKG

SEQ ID NO: 36 - protein, 10 aa, synthetic construct
DSYSNYYFDY

SEQ ID NO: 37 - protein, 17 aa, synthetic construct
KSSQSLLNSRTRKNYLA

SEQ ID NO: 38 - protein, 7 aa, synthetic construct
WASTRES

SEQ ID NO: 39 - protein, 8 aa, synthetic construct
TQSFILRT

SEQ ID NO: 40 - protein, 119 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 41 - protein, 112 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 42 - protein, 6 aa, synthetic construct
GYAFSY

SEQ ID NO: 43 - protein, 8 aa, synthetic construct
FPGDGDTD

SEQ ID NO: 44 - protein, 10 aa, synthetic construct
NVFDGYWLVY

SEQ ID NO: 45 - protein, 16 aa, synthetic construct
RSSKSLLHSNGITYLY

SEQ ID NO: 46 - protein, 7 aa, synthetic construct
QMSNLVS

SEQ ID NO: 47 - protein, 9 aa, synthetic construct
AQNLELPYT

SEQ ID NO: 48 - protein, 119 aa, synthetic construct; identical to SEQ ID NO: 7

SEQ ID NO: 49 - protein, 115 aa, synthetic construct
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV

SEQ ID NO: 50 - protein, 449 aa, synthetic construct
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0232683 A1
(43) Pub. Date: Aug. 13, 2026

(54) THERAPY FOR THE TREATMENT OF NON-HODGKIN LYMPHOMA

(71) Applicants: Celgene Corporation, Princeton, NJ (US); Hoffmann-La Roche Inc., Little Falls, NJ (US); Genentech Inc., South San Francisco, CA (US)

(72) Inventors: David Carlile, Princes Risborough (GB); Linda Maria Lundberg, Binningen (CH); Connie Ma, Belmont, CA (US); Patrick Mueller, Bottmigen (CH); James Christopher Relf, Barnet (GB); Akshay Sudhindra, Jersey City, NJ (US); Donald Lee Yee, Pearland, TX (US)

(73) Assignees: Celgene Corporation, Princeton, NJ (US); Hoffmann-La Roche Inc., Little Falls, NJ (US); Genentech Inc., South San Francisco, CA (US)

(21) Appl. No.: 19/378,979

(22) Filed: Nov. 4, 2025

Related U.S. Application Data
(60) Provisional application No. 63/716,563, filed on Nov. 5, 2024.

(51) Int. Cl. A61K 31/5377; A61K 39/00; A61P 35/00; C07K 16/28

(52) U.S. Cl. CPC A61K 31/5377; A61P 35/00; C07K 16/2809; C07K 16/2887; A61K 2039/505; A61K 2039/54; A61K 2039/545; C07K 2317/31

(57) ABSTRACT

Provided herein are methods of treating and/or managing non-Hodgkin lymphoma, which comprise administering to a patient 2-(2,6-dioxopiperidin-3-yl)-4-((2-fluoro-4-((3-morpholinoazetidin-1-yl)methyl)benzyl)amino)isoindoline-1,3-dione (“Compound A, A-S, or A-R”), or an enantiomer or mixture of enantiomers, a tautomer, an isotopolog, a pharmaceutically acceptable salt, solvate, hydrate, co-crystal, clathrate, or polymorph thereof, in combination with glofitamab or mosunetuzumab.

### CROSS REFERENCE TO RELATED APPLICATIONS

This application claims priority of U.S. Provisional Patent Application No. 63/716,563, filed Nov. 5, 2024, which is hereby incorporated by reference in its entirety.

### SEQUENCE LISTING

The instant application contains a Sequence Listing which has been submitted electronically in XML format and is hereby incorporated by reference in its entirety. Said XML copy, created Nov. 3, 2025, is named 14867-US-NP.xml and is 48,671 bytes in size.

### FIELD

Provided herein are therapies for treating and/or managing non-Hodgkin lymphoma, which comprise administering to a patient 2-(2,6-dioxopiperidin-3-yl)-4-((2-fluoro-4-((3-morpholinoazetidin-1-yl)methyl)benzyl)amino)isoindoline-1,3-dione or an enantiomer or mixture of enantiomers, a tautomer, an isotopolog, a pharmaceutically acceptable salt, solvate, hydrate, co-crystal, clathrate, or polymorph thereof, in combination with glofitamab or mosunetuzumab.

### BACKGROUND

Cancer is characterized primarily by an increase in the number of abnormal cells derived from a given normal tissue, invasion of adjacent tissues by these abnormal cells, or lymphatic or blood-borne spread of malignant cells to regional lymph nodes and metastasis. Clinical data and molecular biologic studies indicate that cancer is a multistep process that begins with minor preneoplastic changes, which may under certain conditions progress to neoplasia. The neoplastic lesion may evolve clonally and develop an increasing capacity for invasion, growth, metastasis, and heterogeneity, especially under conditions in which the neoplastic cells escape the host's immune surveillance. Current cancer therapy may involve surgery, chemotherapy, hormonal therapy and/or radiation treatment to eradicate neoplastic cells in a patient. Recent advances in cancer therapeutics are discussed by Rajkumar et al. in Nature Reviews Clinical Oncology 11, 628-630 (2014).

All of the current cancer therapy approaches pose significant drawbacks for the patient. Surgery, for example, may be contraindicated due to the health of a patient or may be unacceptable to the patient. Additionally, surgery may not completely remove neoplastic tissue. Radiation therapy is only effective when the neoplastic tissue exhibits a higher sensitivity to radiation than normal tissue. Radiation therapy can also often elicit serious side effects. Hormonal therapy is rarely given as a single agent. Although hormonal therapy can be effective, it is often used to prevent or delay recurrence of cancer after other treatments have removed the majority of cancer cells.

With respect to chemotherapy, there are a variety of chemotherapeutic agents available for treatment of cancer. A majority of cancer chemotherapeutics act by inhibiting DNA synthesis, either directly or indirectly by inhibiting the biosynthesis of deoxyribonucleotide triphosphate precursors, to prevent DNA replication and concomitant cell division. Gilman et al., Goodman and Gilman's: The Pharmacological Basis of Therapeutics, Tenth Ed. (McGraw Hill, New York).

Despite availability of a variety of chemotherapeutic agents, chemotherapy has many drawbacks. Stockdale, Medicine, vol. 3, Rubenstein and Federman, eds., ch. 12, sect. 10, 1998. Almost all chemotherapeutic agents are toxic, and chemotherapy causes significant, and often dangerous side effects including severe nausea, bone marrow depression, and immunosuppression. Additionally, even with administration of combinations of chemotherapeutic agents, many tumor cells are resistant or develop resistance to the chemotherapeutic agents. In fact, those cells resistant to the particular chemotherapeutic agents used in the treatment protocol often prove to be resistant to other drugs, even if those agents act by different mechanisms from those of the drugs used in the specific treatment. This phenomenon is referred to as pleiotropic drug or multidrug resistance. Because of the drug resistance, many cancers prove or become refractory to standard chemotherapeutic treatment protocols.

Non-Hodgkin lymphoma (NHL), also known as non-Hodgkin's lymphoma, is the fifth most common cancer for both men and women in the United States (U.S.). An estimated 385,700 patients worldwide were diagnosed with NHL in 2012 and approximately 199,700 patients died as a result of the disease. Torre, L. A. et al. Global cancer statistics, 2012; CA Cancer J. Clin. 65, 87-108 (2015). NHL is a heterogeneous disease comprising diverse B-cell and T-cell lymphoma subtypes that collectively make up approximately 4% of all new cancer cases in the U.S. and account for 3% of cancer-related deaths. Most of NHLs (80% to 90%) are of B-cell origin, and the great majority of the rest are T-cell lymphomas. Common subtypes of NHL include diffuse large B-cell lymphoma (DLBCL), follicular lymphoma (FL), mantle cell lymphoma (MCL), and primary central nervous system lymphoma (PCNSL).

Diffuse large B-cell lymphoma (DLBCL) is the most common subtype of NHL, accounting for up to 30% of newly diagnosed cases, and is clinically classified as an aggressive lymphoma. With the introduction of rituximab plus chemotherapy combination regimens, more than 50% of patients with DLBCL are cured. However, more than 30% of patients in remission will ultimately relapse. For patients who relapse, treatment approaches for second-line DLBCL are less well defined and often are ineffective in achieving long-term disease control. In patients who have received 2 or more lines of therapy and are relapsed and/or refractory and are not candidates for potentially curative therapies due to advanced age or poor performance status, DLBCL remains an incurable disease for which clinical trials are indicated. New therapeutic approaches are still needed.

For follicular lymphoma (FL), the age adjusted incidence rate from 2011-2012 in the U.S. was 3.4 per 100,000. There is no standard treatment for relapsed or refractory (R/R) FL patients. Despite the efforts and advances in front-line treatment, patients with FL continue to experience recurring relapses and require further therapy. The first-line systemic anti-cancer treatments are also considered in second-line therapy; more recently, second-line or later therapy options may include “chemotherapy-free” regimens that are being developed and may become standard of care in the near future. In third-line, patients who fail to respond to a rituximab-containing regimen and have relapsed on or are refractory to additional therapy have limited treatment options and a poor prognosis. There is a high unmet medical need to develop novel treatments for FL patients who fail to respond to standard therapy and whose treatment options for disease remission have been exhausted.

Approximately 6% of all new lymphoma cases each year are mantle cell lymphoma (MCL). The age adjusted incidence rate from 2011-2012 in the U.S. for MCL was 0.8 per 100,000. Despite several available frontline therapies for MCL with prolonged responses, MCL remains an incurable B-cell malignancy. Patients with MCL are often treated with rituximab-chemotherapy combinations, either with or without stem cell transplant consolidation. Relapse is typical, and MCL becomes increasingly resistant to therapy over time.

The age adjusted incidence rate from 2011-2012 in the U.S. for primary central nervous system lymphoma (PCNSL) was 0.3 per 100,000. Despite high response rates with initial high-dose methotrexate (HD-MTX)-based regimens, more than half of the responders relapse. Once PCNSL has relapsed, prognosis remains poor. Novel therapeutic agents with CNS penetration, better efficacy, and tolerable toxicity profile are urgently needed.

There remains a significant need for safe and effective methods of treating, preventing and managing NHL, particularly for NHL that is refractory to standard treatments, such as surgery, radiation therapy, chemotherapy and hormonal therapy, while reducing or avoiding the toxicities and/or side effects associated with conventional therapies.

Citation or identification of any reference in this section of this application is not to be construed as an admission that the reference is prior art to the present application.

### SUMMARY

Provided herein are methods of treating or managing non-Hodgkin lymphoma (NHL). In a first aspect, provided herein is a method of treating or managing non-Hodgkin lymphoma (NHL), comprising administering to a subject having the NHL:
(a) a therapeutically effective amount of a compound, wherein the compound is Compound A

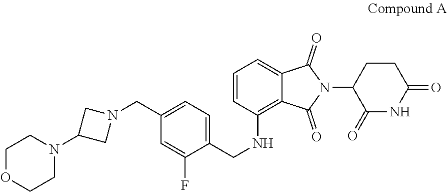 [SMILES: O=C1CCC(N2C(=O)c3cccc(NCc4ccc(CN5CC(N6CCOCC6)C5)cc4F)c3C2=O)C(=O)N1]

or an enantiomer or mixture of enantiomers, a tautomer, an isotopolog, a pharmaceutically acceptable salt, solvate, hydrate, co-crystal, clathrate, or polymorph thereof, in combination with
(b) a therapeutically effective amount of glofitamab or mosunetuzumab.

In a second aspect, provided herein is a compound for use in a method of treating non-Hodgkin lymphoma (NHL), wherein the method comprises administering to a subject having the NHL:
(a) a therapeutically effective amount of the compound, wherein the compound is Compound A

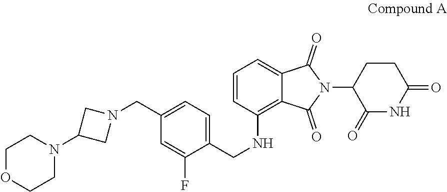 [SMILES: O=C1CCC(N2C(=O)c3cccc(NCc4ccc(CN5CC(N6CCOCC6)C5)cc4F)c3C2=O)C(=O)N1]

or an enantiomer or mixture of enantiomers, a tautomer, an isotopolog, a pharmaceutically acceptable salt, solvate, hydrate, co-crystal, clathrate, or polymorph thereof, in combination with
(b) a therapeutically effective amount of glofitamab or mosunetuzumab.

In a third aspect, provided herein is a bispecific antibody for use in a method of treating non-Hodgkin lymphoma (NHL), wherein the method comprises administering to a subject having the NHL:
(a) a therapeutically effective amount of a compound, wherein the compound is Compound A

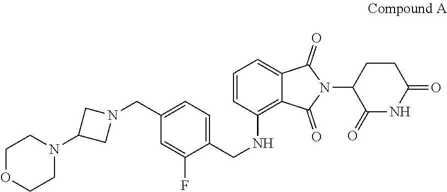 [SMILES: O=C1CCC(N2C(=O)c3cccc(NCc4ccc(CN5CC(N6CCOCC6)C5)cc4F)c3C2=O)C(=O)N1]

or an enantiomer or mixture of enantiomers, a tautomer, an isotopolog, a pharmaceutically acceptable salt, solvate, hydrate, co-crystal, clathrate, or polymorph thereof, in combination with
(b) a therapeutically effective amount of the bispecific antibody, wherein the bispecific antibody is glofitamab or mosunetuzumab.

In a fourth aspect, provided herein is a compound and a bispecific antibody for use in a method of treating non-Hodgkin lymphoma (NHL), wherein the method comprises administering to a subject having the NHL:
(a) a therapeutically effective amount of the compound, wherein the compound is Compound A

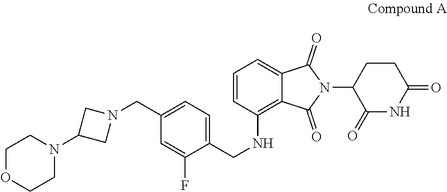 [SMILES: O=C1CCC(N2C(=O)c3cccc(NCc4ccc(CN5CC(N6CCOCC6)C5)cc4F)c3C2=O)C(=O)N1]

or an enantiomer or mixture of enantiomers, a tautomer, an isotopolog, a pharmaceutically acceptable salt, solvate, hydrate, co-crystal, clathrate, or polymorph thereof, in combination with
(b) a therapeutically effective amount of the bispecific antibody, wherein the bispecific antibody is glofitamab or mosunetuzumab.

In a fifth aspect, provided herein is a kit comprising:
(a) Compound A

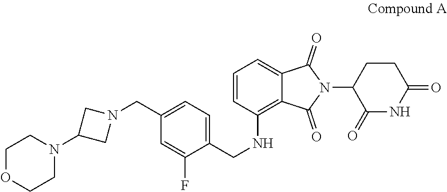 [SMILES: O=C1CCC(N2C(=O)c3cccc(NCc4ccc(CN5CC(N6CCOCC6)C5)cc4F)c3C2=O)C(=O)N1]

or an enantiomer or mixture of enantiomers, a tautomer, an isotopolog, a pharmaceutically acceptable salt, solvate, hydrate, co-crystal, clathrate, or polymorph thereof, and
(b) glofitamab or mosunetuzumab.

In one embodiment of the fifth aspect, the kit is for use in a method of treating non-Hodgkin lymphoma (NHL).

In one embodiment of the first, second, third, fourth or fifth aspect, the compound is Compound A-S

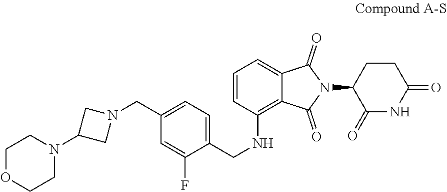 [SMILES: O=C1CC[C@H](N2C(=O)c3cccc(NCc4ccc(CN5CC(N6CCOCC6)C5)cc4F)c3C2=O)C(=O)N1]

or a tautomer, an isotopolog, a pharmaceutically acceptable salt, solvate, hydrate, co-crystal, clathrate, or polymorph thereof. In one embodiment, the compound is a hydrochloride salt of Compound A-S.

In one embodiment of the first, second, third, fourth or fifth aspect, the NHL is B-cell non-Hodgkin lymphoma (BCL). In one embodiment, the NHL is diffuse large B-cell lymphoma (DLBCL) or follicular lymphoma (FL).

In one embodiment of the first, second, third, fourth or fifth aspect, the NHL is relapsed, refractory or resistant. In one embodiment, the NHL is relapsed or refractory. In one embodiment, the NHL is relapsed or refractory to prior anthracycline therapy. In one embodiment, the NHL is relapsed or refractory to prior anti-CD20 therapy. In one embodiment, the NHL is relapsed or refractory to at least two prior therapies.

In one embodiment of the first, second, third, fourth or fifth aspect, the NHL is newly diagnosed.

In one embodiment of the first, second, third, fourth or fifth aspect, the compound is administered orally. In one embodiment, the compound is administered in an amount of about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.11 mg, about 0.12 mg, about 0.13 mg, about 0.14 mg, about 0.15 mg, about 0.16 mg, about 0.17 mg, about 0.18 mg, about 0.19 mg, about 0.2 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1 mg, about 1.05 mg, about 1.1 mg, about 1.15 mg, about 1.2 mg, about 1.25 mg, about 1.3 mg, about 1.35 mg, about 1.4 mg, about 1.45 mg, about 1.5 mg, about 1.55 mg, or about 1.6 mg per day. In one embodiment, the compound is administered in an amount of about 0.1 mg, about 0.2 mg, or about 0.4 mg per day. In one embodiment, the compound is administered once daily for 10 to 14 days followed by 11 to 14 days of rest.

In one embodiment of the first, second, third, fourth or fifth aspect, the bispecific antibody is glofitamab. In one embodiment, the glofitamab is administered intravenously.

In one embodiment of the first, second, third, fourth or fifth aspect, the bispecific antibody is glofitamab and administering the therapeutically effective amount of glofitamab comprises administering glofitamab according to a dosing regimen comprising at least a first dosing cycle (C1) and a second dosing cycle (C2), wherein:
(a) the first dosing cycle (C1) comprises a first dose (C1D1) and a second dose (C1D2) of glofitamab, wherein the C1D1 is about 2.5 mg, and the C1D2 is about 10 mg; and
(b) the second dosing cycle (C2) comprises a single dose (C2D1) of glofitamab, wherein the C2D1 is about 30 mg.

In one embodiment, the first dosing cycle (C1) further comprises a single dose (OD1) of obinutuzumab, wherein the OD1 is about 1,000 mg.

In one embodiment, the length of the first dosing cycle (C1) is about 21 days, and optionally the dosing regimen comprises administering to the subject the C1D1 and the C1D2 on or about days 8 and 15, respectively, of the first dosing cycle (C1) or optionally the dosing regimen comprises administering to the subject the OD1, the C1D1 and the C1D2 on or about days 1, 8 and 15, respectively, of the first dosing cycle (C1). In one embodiment, the length of the second dosing cycle (C2) is about 21 days, and optionally the dosing regimen comprises administering to the subject the C2D1 on or about day 1 of the second dosing cycle (C2).

In one embodiment, the dosing regimen comprises one or more additional dosing cycles, such as one to ten additional dosing cycles (C3-C12), or such as ten additional dosing cycles (C3-C12). In one embodiment, the length of the additional dosing cycles is about 21 days. In one embodiment, the additional dosing cycles comprise a single dose (AD1) of glofitamab, wherein the AD1 is about 30 mg, and optionally the dosing regimen comprises administering to the subject the AD1 on or about day 1 of the additional dosing cycles.

In one embodiment, the compound is administered on days 1 to 10 of:
the additional dosing cycles; or
the second dosing cycle (C2); or
the second dosing cycle (C2) and the additional dosing cycles; or
the first dosing cycle (C1) and the second dosing cycle (C2); or
the first dosing cycle (C1), the second dosing cycle (C2) and the additional dosing cycles.

In one embodiment, the compound is administered on days 1 to 10 of the first dosing cycle (C1) and/or the second dosing cycle (C2) and/or the additional dosing cycles in an amount of about 0.1 mg, about 0.2 mg or about 0.4 mg per day.

In one embodiment of the first, second, third, fourth or fifth aspect, the bispecific antibody is glofitamab and administering the therapeutically effective amount of glofitamab comprises administering glofitamab intravenously according to a dosing regimen comprising a first dosing cycle (C1), a second dosing cycle (C2) and one to ten additional dosing cycles (C3-C12), wherein:
(a) the length of the first dosing cycle (C1) is about 21 days and the first dosing cycle (C1) comprises on day 1 a single dose (OD1) of obinutuzumab, on day 8 a first dose (C1D1) of glofitamab and on day 15 a second dose (C1D2) of glofitamab, wherein the OD1 is about 1,000 mg, the C1D1 is about 2.5 mg, and the C1D2 is about 10 mg;
(b) the length of the second dosing cycle (C2) is about 21 days and the second dosing cycle (C2) comprises on day 1 a single dose (C2D1) of glofitamab, wherein the C2D1 is about 30 mg; and
(c) the length of the one to ten additional dosing cycles (C3-C12) is about 21 days and the one to ten additional dosing cycles (C3-C12) comprise on day 1 a single dose (AD1) of glofitamab, wherein the AD1 is about 30 mg;
wherein the compound is administered orally in an amount of about 0.1 mg, about 0.2 mg or about 0.4 mg per day on days 1 to 10 of (i) the one to ten additional dosing cycles (C3-C12), or (ii) the second dosing cycle (C2) and the one to ten additional dosing cycles (C3-C12), or (iii) the first dosing cycle (C1), the second dosing cycle (C2) and the one to ten additional dosing cycles (C3-C12).

In one embodiment of the first, second, third, fourth or fifth aspect, the bispecific antibody is mosunetuzumab. In one embodiment, the mosunetuzumab is administered subcutaneously.

In one embodiment of the first, second, third, fourth or fifth aspect, the bispecific antibody is mosunetuzumab and administering the therapeutically effective amount of mosunetuzumab comprises administering mosunetuzumab according to a dosing regimen comprising at least a first dosing cycle (C1) and a second dosing cycle (C2), wherein:
(a) the first dosing cycle (C1) comprises a first dose (C1D1), a second dose (C1D2), and a third dose (C1D3) of mosunetuzumab, wherein the C1D1 is about 5 mg, the C1D2 is about 45 mg, and the C1D3 is about 45 mg; and
(b) the second dosing cycle (C2) comprises a single dose (C2D1) of mosunetuzumab, wherein the C2D1 is about 45 mg.

In one embodiment, the length of the first dosing cycle (C1) is about 21 days, and optionally the dosing regimen comprises administering to the subject the C1D1, the C1D2 and the C1D3 on or about days 1, 8 and 15, respectively, of the first dosing cycle (C1). In one embodiment, the length of the second dosing cycle (C2) is about 28 days, and optionally the dosing regimen comprises administering to the subject the C2D1 on or about day 1 of the second dosing cycle (C2).

In another embodiment, the length of the first dosing cycle (C1) is about 28 days, and optionally the dosing regimen comprises administering to the subject the C1D1, the C1D2 and the C1D3 on or about days 1, 8 and 15, respectively, of the first dosing cycle (C1). In one embodiment, the length of the second dosing cycle (C2) is about 28 days, and optionally the dosing regimen comprises administering to the subject the C2D1 on or about day 1 of the second dosing cycle (C2).

In one embodiment, the dosing regimen comprises one or more additional dosing cycles, such as one to ten additional dosing cycles (C3-C12), or such as ten additional dosing cycles (C3-C12). In one embodiment, the length of the additional dosing cycles is about 28 days. In one embodiment, the additional dosing cycles comprise a single dose (AD1) of mosunetuzumab, wherein the AD1 is about 45 mg, and optionally the dosing regimen comprises administering to the subject the AD1 on or about day 1 of the additional dosing cycles.

In one embodiment, the compound is administered on days 1 to 14 of:
the second dosing cycle (C2); or
the second dosing cycle (C2) and the additional dosing cycles; or
the first dosing cycle (C1) and the second dosing cycle (C2); or
the first dosing cycle (C1), the second dosing cycle (C2) and the additional dosing cycles.

In one embodiment, the compound is administered on days 1 to 14 of the first dosing cycle (C1) and/or the second dosing cycle (C2) and/or the additional dosing cycles in an amount of about 0.1 mg, about 0.2 mg or about 0.4 mg per day.

In one embodiment of the first, second, third, fourth or fifth aspect, the bispecific antibody is mosunetuzumab and administering the therapeutically effective amount of mosunetuzumab comprises administering mosunetuzumab subcutaneously according to a dosing regimen comprising a first dosing cycle (C1) and one to eleven additional dosing cycles (C2-C12), wherein:
(a) the length of the first dosing cycle (C1) is about 21 days and the first dosing cycle (C1) comprises on day 1 a first dose (C1D1), on day 8 a second dose (C1D2), and on day 15 a third dose (C1D3) of mosunetuzumab, wherein the C1D1 is about 5 mg, the C1D2 is about 45 mg, and the C1D3 is about 45 mg; and
(b) the length of the one to eleven additional dosing cycles (C2-C12) is about 28 days and the one to eleven additional dosing cycles (C2-C12) comprise on day 1 a single dose (AD1) of mosunetuzumab, wherein the AD1 is about 45 mg;
wherein the compound is administered orally in an amount of about 0.1 mg, about 0.2 mg or about 0.4 mg per day on days 1 to 14 of the one to eleven additional dosing cycles (C2-C12).

In one embodiment of the first, second, third, fourth or fifth aspect, the bispecific antibody is mosunetuzumab and administering the therapeutically effective amount of mosunetuzumab comprises administering mosunetuzumab subcutaneously according to a dosing regimen comprising a first dosing cycle (C1) and one to eleven additional dosing cycles (C2-C12), wherein:
(a) the length of the first dosing cycle (C1) is about 28 days and the first dosing cycle (C1) comprises on day 1 a first dose (C1D1), on day 8 a second dose (C1D2), and on day 15 a third dose (C1D3) of mosunetuzumab, wherein the C1D1 is about 5 mg, the C1D2 is about 45 mg, and the C1D3 is about 45 mg; and
(b) the length of the one to eleven additional dosing cycles (C2-C12) is about 28 days and the one to eleven additional dosing cycles (C2-C12) comprise on day 1 a single dose (AD1) of mosunetuzumab, wherein the AD1 is about 45 mg;
wherein the compound is administered orally in an amount of about 0.1 mg, about 0.2 mg or about 0.4 mg per day on days 1 to 14 of the first dosing cycle (C1) and the one to eleven additional dosing cycles (C2-C12).

### BRIEF DESCRIPTION OF THE FIGURES

FIG. 1 illustrates the study design in Example 1.

FIGS. 2 and 3 illustrate the exemplary dosing schedules for mosunetuzumab and Compound A-S.

FIG. 4 to 6 illustrate the exemplary dosing schedules for glofitamab and Compound A-S.

FIG. 7 is a schematic of the molecule structure of glofitamab.

FIG. 8 to 10 illustrate the preliminary efficacy results from Example 1 as discussed in Example 2, showing respectively best overall response and DOR, best overall response by prior CAR T-cell therapy, and best overall response by dose histology.

### DETAILED DESCRIPTION

### Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

The use of the word “a” or “an” when used in conjunction with the term “comprising” in the claims and/or the description can mean “one”, but it is also consistent with the meaning of “one or more”, “at least one” and “one or more than one”.

As used herein, the terms “comprising” and “including” can be used interchangeably. The terms “comprising” and “including” are to be interpreted as specifying the presence of the stated features or components as referred to, but do not preclude the presence of additional features or components. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: A; B; C; A and B; A and C; B and C; A, B and C. An exception to this definition will occur only when a combination of elements, functions, steps or acts are in some way inherently mutually exclusive.

As used herein, and unless otherwise specified, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means that the numeric value or range of values may vary within 25%, 20%, 15%, 10%, 9%, 8%, 7%, 6%, 5%, 4%, 3%, 2%, 1.5%, 1%, 0.5%, or 0.25% of the recited value or range of values. In one embodiment, the term “about” refers to a value that is no more than 10% above or below the value being modified by the term. For example, the term “about 10 mg/m²” means a range of from 9 mg/m² to 11 mg/m².

The term “optional” or “optionally” means that the subsequently described circumstance may or may not occur, so that the description includes instances wherein the circumstance occurs, and instances wherein the circumstance does not occur.

Unless specifically stated otherwise, where a compound may assume alternative tautomeric, regioisomeric and/or stereoisomeric forms, all alternative isomers are intended to be encompassed within the scope of the claimed subject matter. For example, where a compound has one or more chiral centers, it can occur as racemate, individual enantiomer or diastereomer, or mixture thereof; it is intended that all such isomeric forms be encompassed herein. Where a compound can have one or more tautomeric forms, it is intended that all tautomers be encompassed herein.

Thus, the compounds provided herein may be enantiomerically pure, or be stereoisomeric or diastereomeric mixtures. As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A typical stereomerically pure compound comprises greater than about 80% by weight of one stereoisomer of the compound and less than about 20% by weight of the other stereoisomers of the compound, in one embodiment greater than about 90% by weight of one stereoisomer of the compound and less than about 10% by weight of the other stereoisomers of the compound, in one embodiment greater than about 95% by weight of one stereoisomer of the compound and less than about 5% by weight of the other stereoisomers of the compound, and in one embodiment greater than about 97% by weight of one stereoisomer of the compound and less than about 3% by weight of the other stereoisomers of the compound. A stereomerically pure Compound A, A-S, or A-R used herein comprises greater than about 85% by weight of one stereoisomer of the compound, in one embodiment greater than about 90% by weight of one stereoisomer of the compound, in one embodiment greater than about 95% by weight of one stereoisomer of the compound, and in one embodiment greater than about 97% by weight of one stereoisomer of the compound. As used herein and unless otherwise indicated, the term “stereomerically enriched” means a composition that comprises greater than about 60% by weight of one stereoisomer of a compound, in one embodiment greater than about 70% by weight of one stereoisomer of a compound, and in one embodiment greater than about 80% by weight of one stereoisomer of a compound. As used herein and unless otherwise indicated, the term “enantiomerically pure” means a stereomerically pure composition of a compound having one chiral center. Similarly, the term “enantiomerically enriched” means a stereomerically enriched composition of a compound having one chiral center. As used herein, stereoisomeric or diastereomeric mixtures means a composition that comprises more than one stereoisomer of a compound. A typical stereoisomeric mixture of a compound comprises about 50% by weight of one stereoisomer of the compound and about 50% by weight of the other stereoisomers of the compound, or comprises greater than about 50% by weight of one stereoisomer of the compound and less than about 50% by weight of the other stereoisomers of the compound, or comprises greater than about 45% by weight of one stereoisomer of the compound and less than about 55% by weight of the other stereoisomers of the compound, or comprises greater than about 40% by weight of one stereoisomer of the compound and less than about 60% by weight of the other stereoisomers of the compound, or comprises greater than about 35% by weight of one stereoisomer of the compound and less than about 65% by weight of the other stereoisomers of the compound.

It is to be understood that the compounds provided herein may contain chiral centers. Such chiral centers may be of either the (R) or (S) configuration, or may be a mixture thereof. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

Optically active (+) and (−), (R)- and (S)-, or (D)- and (L)-isomers may be prepared using chiral synthons or chiral reagents, or resolved using conventional techniques, such as chromatography on a chiral stationary phase.

A compound provided herein can contain unnatural proportions of atomic isotopes at one or more of its atoms. For example, the compound may be radiolabeled with radioactive isotopes, such as for example tritium (³H) or carbon-14 (¹⁴C), or may be isotopically enriched, such as with deuterium (D or ²H), carbon-13 (¹³C), or nitrogen-15 (¹⁵N). As used herein, an “isotopolog” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., non-Hodgkin lymphoma therapeutic agents, research reagents, e.g., binding assay reagents, and diagnostic agents, e.g., in vivo imaging agents. All isotopic variations of the compounds described herein, whether radioactive or not, are intended to be encompassed within the scope of the embodiments described herein. In some embodiments, provided herein are isotopologues of the compounds, for example, the isotopologues are deuterium, carbon-13 (¹³C), and/or nitrogen-15 (¹⁵N) enriched compounds. As used herein, “deuterated”, means a compound wherein at least one hydrogen (H) has been replaced by deuterium (D or ²H), that is, the compound is enriched in deuterium in at least one position. In some embodiments, isotopologues provided herein are deuterium enriched compounds. In some embodiments, isotopologues provided herein are deuterium enriched compounds, where the deuteration occurs on the chiral center.

In the description herein, if there is any discrepancy between a chemical name and a chemical structure, the structure controls.

As used herein and unless otherwise indicated, the term “solvate” means a compound provided herein or a salt thereof, that further includes a stoichiometric or non-stoichiometric amount of solvent bound by non-covalent intermolecular forces. Where the solvent is water, the solvate is a hydrate.

As used herein and unless otherwise specified, the term “pharmaceutically acceptable salt” encompasses non-toxic acid and base addition salts of the compound to which the term refers. Acceptable non-toxic acid addition salts include those derived from organic and inorganic acids known in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like. Others are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N′-dibenzylethylenediamine, chloroprocaine, choline, diethanolamine, ethylenediamine, meglumine (N-methylglucamine), lysine, and procaine.

It is understood that, independently of stereomerical or isotopic composition, each compound provided herein can be provided in the form of any of the pharmaceutically acceptable salts provided herein. Equally, it is understood that the isotopic composition may vary independently from the stereomerical composition of each compound provided herein. Further, the isotopic composition, while being restricted to those elements present in the respective compound or salt thereof, may otherwise vary independently from the selection of the pharmaceutically acceptable salt of the respective compound.

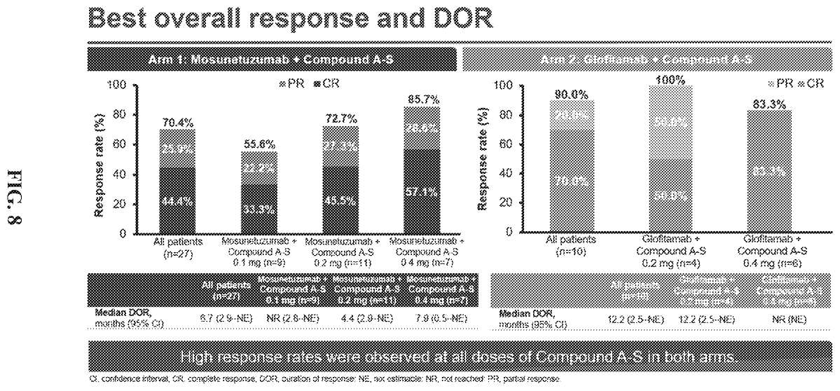

“Glofitamab” (WHO Drug Information (International Nonproprietary Names for Pharmaceutical Substances), Recommended INN: List 83, 2020, vol. 34, no. 1, p. 39; also known as CD20-TCB, RO7082859, or RG6026; CAS #: 2229047-91-8) is a T-cell-engaging bispecific (TCB) full-length antibody with a 2:1 molecular configuration for bivalent binding to CD20 on B-cells and monovalent binding to CD3, particularly the CD3 epsilon chain (CD3ε), on T-cells. Its CD3-binding region is fused to one of the CD20-binding regions in a head to-tail fashion via a flexible linker. This structure endows glofitamab with superior in vitro potency versus other CD20-CD3 bispecific antibodies with a 1:1 configuration and leads to profound antitumor efficacy in preclinical DLBCL models. Glofitamab comprises an engineered, heterodimeric Fc region with completely abolished binding to FcγRs and C1q. By simultaneously binding to human CD20-expressing tumor cells and to the CD3ε of the T-cell receptor (TCR) complex on T-cells, it induces tumor cell lysis, in addition to T-cell activation, proliferation and cytokine release. Lysis of B-cells mediated by glofitamab is CD20-specific and does not occur in the absence of CD20 expression or in the absence of simultaneous binding (cross-linking) of T-cells to CD20-expressing cells. In addition to killing, T-cells undergo activation due to CD3 cross-linking, as detected by an increase in T-cell activation markers (CD25 and CD69), cytokine release (IFNγ, TNFα, IL-2, IL-6, IL-10), cytotoxic granule release (Granzyme B) and T-cell proliferation. Further discussion of glofitamab can be found, for example, in PCT publication WO2022/228705, the entirety of which is incorporated herein by reference. A schematic of the molecule structure of glofitamab is depicted in FIG. 7. The sequences of glofitamab are summarized in Table 1.

TABLE 1
Sequence IDs for Glofitamab

| SEQ ID NO: | Description | SEQ ID NO: | Description |
|---|---|---|---|
| CD3 Heavy Chain | | CD3 Light Chain | |
| 9 | HVR-H1 (Kabat) | 12 | HVR-L1 (Kabat) |
| 10 | HVR-H2 (Kabat) | 13 | HVR-L2 (Kabat) |
| 11 | HVR-H3 (Kabat) | 14 | HVR-L3 (Kabat) |
| 15 | VH | 16 | VL |
| SEQ ID NO: | Description | SEQ ID NO: | Description |
| CD20 Heavy Chain | | CD20 Light Chain | |
| 1 | HVR-H1 (Kabat) | 4 | HVR-L1 (Kabat) |
| 2 | HVR-H2 (Kabat) | 5 | HVR-L2 (Kabat) |
| 3 | HVR-H3 (Kabat) | 6 | HVR-L3 (Kabat) |
| 7 | VH | 8 | VL |

Full-length antibody

| | | | |
|---|---|---|---|
| 17 | HC-knob | 18 | HC-hole |
| 19 | LC-CD3 | 20 | LC-CD20 |

Glofitamab has (1) an anti-CD20 arm comprising the heavy chain and light chain sequences of SEQ ID NOs: 7 and 8, respectively; and (2) an anti-CD3 arm comprising the heavy chain and light chain sequences of SEQ ID NOs: 15 and 16, respectively. It is a T-cell activating bispecific antigen binding molecule, wherein the molecule comprises a heavy chain of SEQ ID NO: 17, a heavy chain of SEQ ID NO: 18, a light chain of SEQ ID NO: 20, and a light chain of SEQ ID NO: 19. It is a T-cell activating bispecific antigen binding molecule, wherein the molecule comprises a heavy chain of SEQ ID NO: 17, a heavy chain of SEQ ID NO: 18, two light chains of SEQ ID NO: 20, and a light chain of SEQ ID NO: 19; and wherein the heavy and light chains are assembled to form a first Fab molecule which specifically binds to CD20, a second Fab molecule which specifically binds to CD3 and a third Fab molecule which specifically binds to CD20, and a Fc domain composed of a first and a second subunit capable of stable association.

“Mosunetuzumab” has the International Nonproprietary Names for Pharmaceutical Substances (INN) List 117 (WHO Drug Information, vol. 31, no. 2, 2017, p. 303), or CAS Registry No. 1905409-39-3, and is an anti-CD20/anti-CD3 bispecific antibody having (1) an anti-CD20 arm comprising the heavy chain and light chain sequences of SEQ ID NOs: 22 and 23, respectively; and (2) an anti-CD3 arm comprising the heavy chain and light chain sequences of SEQ ID NOs: 24 and 25, respectively. The amino acid sequences of mosunetuzumab are summarized in Table 2 below.

TABLE 2
Sequence IDs for Mosunetuzumab

| CD3 Arm | | CD20 Arm | |
|---|---|---|---|
| SEQ ID NO: | Description | SEQ ID NO: | Description |
| 34 | CD3 HVR-H1 | 26 | CD20 HVR-H1 |
| 35 | CD3 HVR-H2 | 27 | CD20 HVR-H2 |
| 36 | CD3 HVR-H3 | 28 | CD20 HVR-H3 |
| 37 | CD3 HVR-L1 | 29 | CD20 HVR-L1 |
| 38 | CD3 HVR-L2 | 30 | CD20 HVR-L2 |
| 39 | CD3 HVR-L3 | 31 | CD20 HVR-L3 |
| 40 | CD3 VH | 32 | CD20 VH |
| 41 | CD3 VL | 33 | CD20 VL |
| 24 | CD3 heavy chain | 22 | CD20 heavy chain |
| 25 | CD3 light chain | 23 | CD20 light chain |

Mosunetuzumab may be produced using recombinant methods and compositions, for example, as described in U.S. Pat. No. 4,816,567, the entirety of which is incorporated herein by reference. Glofitamab may similarly be produced using recombinant methods and compositions.

“Obinutuzumab” is an IgG1 antibody that binds human CD20. Obinutuzumab (also known as GA101 or RO5072759) comprises (1) an HVR-H1 comprising the amino acid sequence of SEQ ID NO: 42, an HVR-H2 comprising the amino acid sequence of SEQ ID NO: 43, an HVR-H3 comprising the amino acid sequence of SEQ ID NO: 44, an HVR-L1 comprising the amino acid sequence of SEQ ID NO: 45, an HVR-L2 comprising the amino acid sequence of SEQ ID NO: 46, and an HVR-L3 comprising the amino acid sequence of SEQ ID NO: 47; (2) an antibody comprising a VH domain comprising the amino acid sequence of SEQ ID NO: 48 and a VL domain comprising the amino acid sequence of SEQ ID NO: 49; and (3) an antibody comprising the amino acid sequence of SEQ ID NO: 50 and the amino acid sequence of SEQ ID NO: 21 (recommended INN, WHO Drug Information, Vol. 26, No. 4, 2012, p. 453). As used herein, obinutuzumab is synonymous for GA101 or RO5072759. This replaces all previous versions (e.g. Vol. 25, No. 1, 2011, p. 75-76), and is formerly known as afutuzumab (recommended INN, WHO Drug Information, Vol. 23, No. 2, 2009, p. 176; Vol. 22, No. 2, 2008, p. 124). The amino acid sequences of obinutuzumab are summarized in Table 3 below.

TABLE 3
Sequence IDs for Obinutuzumab

| SEQ ID NO: | Sequence | Description |
|---|---|---|
| 42 | GYAFSY | HVR-H1 |
| 43 | FPGDGDTD | HVR-H2 |
| 44 | NVFDGYWLVY | HVR-H3 |
| 45 | RSSKSLLHSN GITYLY | HVR-L1 |
| 46 | QMSNLVS | HVR-L2 |
| 47 | AQNLELPYT | HVR-L3 |
| 48 | QVQLVQSGAE VKKPGSSVKV SCKASGYAFS | VH |
| | YSWINWVRQA PGQGLEWMGR IFPGDGDTDY 60 | |
| | NGKFKGRVTI TADKSTSTAY MELSSLRSED | |
| | TAVYYCARNV FDGYWLVYWG QGTLVTVSS 119 | |
| 49 | DIVMTQTPLS LPVTPGEPAS ISCRSSKSLL | VL |
| | HSNGITYLYW YLQKPGQSPQ LLIYQMSNLV 60 | |
| | SGVPDRESGS GSGTDFTLKI SRVEAEDVGV | |
| | YYCAQNLELP YTFGGGTKVE IKRTV 115 | |
| 50 | QVQLVQSGAE VKKPGSSVKV SCKASGYAFS | HC |
| | YSWINWVRQA PGQGLEWMGR IFPGDGDTDY 60 | |
| | NGKFKGRVTI TADKSTSTAY MELSSLRSED | |
| | TAVYYCARNV FDGYWLVYWG QGTLVTVSSA 120 | |
| | STKGPSVFPL APSSKSTSGG TAALGCLVKD | |
| | YFPEPVTVSW NSGALTSGVH TFPAVLQSSG 180 | |
| | LYSLSSVVTV PSSSLGTQTY ICNVNHKPSN | |
| | TKVDKKVEPK SCDKTHTCPP CPAPELLGGP 240 | |
| | SVFLFPPKPK DTLMISRTPE VTCVVVDVSH | |
| | EDPEVKENWY VDGVEVHNAK TKPREEQYNS 300 | |
| | TYRVVSVLTV LHQDWLNGKE YKCKVSNKAL | |
| | PAPIEKTISK AKGQPREPQV YTLPPSRDEL 360 | |
| | TKNQVSLTCL VKGFYPSDIA VEWESNGQPE | |
| | NNYKTTPPVL DSDGSFFLYS KLTVDKSRWQ 420 | |
| | QGNVFSCSVM HEALHNHYTQ KSLSLSPGK 449 | |
| 21 | DIVMTQTPLS LPVTPGEPAS ISCRSSKSLL | LC |
| | HSNGITYLYW YLQKPGQSPQ LLIYQMSNLV 60 | |
| | SGVPDRFSGS GSGTDFTLKI SRVEAEDVGV | |
| | YYCAQNLELP YTFGGGTKVE IKRTVAAPSV 120 | |
| | FIFPPSDEQL KSGTASVVCL LNNFYPREAK | |
| | VQWKVDNALQ SGNSQESVTE QDSKDSTYSL 180 | |
| | SSTLTLSKAD YEKHKVYACE VTHQGLSSPV | |
| | TKSFNRGEC 219 | |

It is known in the art that glutamine and glutamate at the N termini of recombinant monoclonal antibodies can cyclize spontaneously to pyroglutamate in vitro. In some embodiments, for each sequence disclosed herein that contains an N-terminal glutamine and/or glutamate, the corresponding sequence with N-terminal pyroglutamate residues is also contemplated.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen binding activity.

The terms “full length antibody”, “intact antibody” and “whole antibody” are used herein interchangeably to refer to an antibody having a structure substantially similar to a native antibody structure or having heavy chains that contain an Fc region as defined herein.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2, diabodies, linear antibodies, single-chain antibody molecules (e.g., scFv), and multispecific antibodies formed from antibody fragments. The term “antibody fragment” as used herein also encompasses single-domain antibodies.

The term “immunoglobulin molecule” refers to a protein having the structure of a naturally occurring antibody. For example, immunoglobulins of the IgG class are heterotetrameric glycoproteins of about 150,000 Daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The heavy chain of an immunoglobulin may be assigned to one of five classes, called α (IgA), δ (IgD), ε (IgE), γ (IgG), or μ (IgM), some of which may be further divided into subclasses, e.g., γ1 (IgG1), γ2 (IgG2), γ3 (IgG3), γ4 (IgG4), α1 (IgA1) and α2 (IgA2). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “antigen binding domain” refers to the part of an antibody that comprises the area which specifically binds to and is complementary to part or all of an antigen. An antigen binding domain may be provided by, for example, one or more antibody variable domains (also called antibody variable regions). Preferably, an antigen binding domain comprises an antibody light chain variable region (VL) and an antibody heavy chain variable region (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol., 5: 368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Natl. Acad. Sci. USA, 103:3557-3562 (2006) regarding human antibodies generated via a human B-cell hybridoma technology.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ respectively.

The term IgG “isotype” or “subclass” as used herein is meant any of the subclasses of immunoglobulins defined by the chemical and antigenic characteristics of their constant regions.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain (also referred to herein as a “cleaved variant heavy chain”). This may be the case where the final two C-terminal amino acids of the heavy chain are glycine (G446) and lysine (K447, EU numbering). Therefore, the C-terminal lysine (Lys447), or the C-terminal glycine (Gly446) and lysine (K447), of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991 (see also above). A “subunit” of an Fc domain as used herein refers to one of the two polypeptides forming the dimeric Fc domain, i.e., a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e., the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g., antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89).

The term “effector functions” when used in reference to antibodies refer to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g., B-cell receptors), and B-cell activation.

As used herein, the term “effector cells” refers to a population of lymphocytes that display effector moiety receptors, e.g., cytokine receptors, and/or Fc receptors on their surface through which they bind an effector moiety, e.g., a cytokine, and/or an Fc region of an antibody and contribute to the destruction of target cells, e.g., tumor cells. Effector cells may for example mediate cytotoxic or phagocytic effects. Effector cells include, but are not limited to, effector T-cells such as CD8+ cytotoxic T-cells, CD4+ helper T-cells, γδ T-cells, NK cells, lymphokine-activated killer (LAK) cells and macrophages/monocytes.

As used herein, the terms “engineer”, “engineered” and “engineering” are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches. “Engineering”, particularly with the prefix “glyco-”, as well as the term “glycosylation engineering” include metabolic engineering of the glycosylation machinery of a cell, including genetic manipulations of the oligosaccharide synthesis pathways to achieve altered glycosylation of glycoproteins expressed in cells. Furthermore, glycosylation engineering includes the effects of mutations and cell environment on glycosylation. In one embodiment, the glycosylation engineering is an alteration in glycosyltransferase activity. In a particular embodiment, the engineering results in altered glucosaminyltransferase activity and/or fucosyltransferase activity. Glycosylation engineering can be used to obtain a “host cell having increased GnTIII activity” (e.g., a host cell that has been manipulated to express increased levels of one or more polypeptides having β(1,4)-N-acetylglucosaminyltransferase III (GnTIII) activity), a “host cell having increased ManII activity” (e.g., a host cell that has been manipulated to express increased levels of one or more polypeptides having α-mannosidase II (ManII) activity), or a “host cell having decreased α(1,6) fucosyltransferase activity” (e.g., a host cell that has been manipulated to express decreased levels of α(1,6) fucosyltransferase).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Native antibodies” refer to naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

As used herein, the terms “first”, “second”, “third” etc. with respect to antigen binding moieties or domains, are used for convenience of distinguishing when there is more than one of each type of moiety or domain. Use of these terms is not intended to confer a specific order or orientation unless explicitly so stated.

The terms “multispecific” and “bispecific” mean that the antigen binding molecule is able to specifically bind to at least two distinct antigenic determinants. Typically, a bispecific antigen binding molecule comprises two antigen binding sites, each of which is specific for a different antigenic determinant. In certain embodiments, a bispecific antigen binding molecule is capable of simultaneously binding two antigenic determinants, particularly two antigenic determinants expressed on two distinct cells.

The term “valent” or “valency” as used herein denotes the presence of a specified number of antigen binding sites in an antigen binding molecule. As such, the term “monovalent binding to an antigen” denotes the presence of one (and not more than one) antigen binding site specific for the antigen in the antigen binding molecule.

An “antigen binding site” refers to the site, i.e., one or more amino acid residues, of an antigen binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site.

An “activating T-cell antigen” as used herein refers to an antigenic determinant expressed by a T lymphocyte, particularly a cytotoxic T lymphocyte, which is capable of inducing or enhancing T-cell activation upon interaction with an antigen binding molecule. Specifically, interaction of an antigen binding molecule with an activating T-cell antigen may induce T-cell activation by triggering the signaling cascade of the T-cell receptor complex. An exemplary activating T-cell antigen is CD3. In a particular embodiment the activating T-cell antigen is CD3, particularly the epsilon subunit of CD3 (see UniProt no. P07766 (version 130), NCBI Ref Seq no. NP_000724.1, for the human sequence; or UniProt no. Q95LI5 (version 49), NCBI GenBank no. BAB71849.1, for the cynomolgus [Macaca fascicularis] sequence).

“T-cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. The T-cell activating therapeutic agents used in the present invention are capable of inducing T-cell activation. Suitable assays to measure T-cell activation are known in the art described herein.

A “target cell antigen” as used herein refers to an antigenic determinant presented on the surface of a target cell, for example a cell in a tumor such as a cancer cell or a cell of the tumor stroma. In a particular embodiment, the target cell antigen is CD20, particularly human CD20 (see UniProt no. P11836).

A “B-cell antigen” as used herein refers to an antigenic determinant presented on the surface of a B lymphocyte, particularly a malignant B lymphocyte (in that case the antigen also being referred to as “malignant B-cell antigen”).

A “T-cell antigen” as used herein refers to an antigenic determinant presented on the surface of a T lymphocyte, particularly a cytotoxic T lymphocyte.

A “Fab molecule” refers to a protein consisting of the VH and CH1 domain of the heavy chain (the “Fab heavy chain”) and the VL and CL domain of the light chain (the “Fab light chain”) of an immunoglobulin.

By a “crossover” Fab molecule (also termed “Crossfab”) is meant a Fab molecule wherein the variable domains or the constant domains of the Fab heavy and light chain are exchanged (i.e., replaced by each other), i.e., the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain VH is referred to herein as the “heavy chain” of the (crossover) Fab molecule.

In contrast thereto, by a “conventional” Fab molecule is meant a Fab molecule in its natural format, i.e., comprising a heavy chain composed of the heavy chain variable and constant domains (VH-CH1, in N- to C-terminal direction), and a light chain composed of the light chain variable and constant domains (VL-CL, in N- to C-terminal direction).

As used herein, the terms “specifically binds”, “specifically recognizes”, “immunospecifically binds”, “selectively binds”, “immunospecifically recognizes” and “immunospecific” are analogous terms in the context of antibodies and refer to molecules that bind to an antigen (e.g., epitope) as such binding is understood by one skilled in the art. In some embodiments, “specifically binds” means, for instance that a polypeptide or molecule interacts more frequently, more rapidly, with greater duration, with greater affinity, or with some combination of the above to the epitope, protein, or target molecule than with alternative substances, including related and unrelated proteins. For example, a molecule that specifically binds to an antigen may bind to other peptides or polypeptides, generally with lower affinity as determined by, e.g., immunoassays, BIACORE™, KinExA 3000 instrument (Sapidyne Instruments, Boise, ID), the OctetQK384 system (ForteBio, Menlo Park, CA), or other assays known in the art. In some embodiments, an antibody or antigen-binding domain binds to or specifically binds to an antigen when it binds to the antigen with higher affinity than to any cross-reactive antigen as determined using experimental techniques, such as radioimmunoassays (RIAs) and enzyme linked immunosorbent assays (ELISAs). Typically a specific or selective reaction will be at least twice background signal or noise and may be more than 10 times background. See, e.g., Fundamental Immunology 332-36 (Paul ed., 2d ed. 1989) for a discussion regarding binding specificity. In some embodiments, the extent of binding of an antibody or antigen-binding domain to a “non-target” protein is less than about 10% of the binding of the antibody or antigen-binding domain to its particular target antigen, for example, as determined by fluorescence activated cell sorting (FACS) analysis or RIAs. In some embodiments, molecules that specifically bind to an antigen bind to the antigen with a Ka that is at least 2 logs, 2.5 logs, 3 logs, 4 logs or greater than the Ka when the molecules bind to another antigen. In some embodiments, molecules that specifically bind to an antigen do not cross react with other proteins. In another specific embodiment, molecules that specifically bind to an antigen do not cross react with other proteins. In some embodiments “specifically binds” means, for instance, that a polypeptide or molecule binds a protein or target with a K_{D} of about 0.1 mM or less, but more usually less than about 1 μM. In some embodiments, “specifically binds” means that a polypeptide or molecule binds a target with a K_{D} of at least about 0.1p M or less, at least about 0.01 μM or less, or at least about 1 nM or less. Because of the sequence identity between homologous proteins in different species, specific binding can include a polypeptide or molecule that recognizes a protein or target in more than one species. Likewise, because of homology within certain regions of polypeptide sequences of different proteins, specific binding can include a polypeptide or molecule that recognizes more than one protein or target. It is understood that, in some embodiments, a polypeptide or molecule that specifically binds a first target may or may not specifically bind a second target. As such, “specific binding” does not necessarily require (although it can include) exclusive binding, e.g., binding to a single target. Thus, a polypeptide or molecule can, in some embodiments, specifically bind more than one target. In some embodiments, multiple targets can be bound by the same antigen-binding site on the polypeptide or molecule. For example, an antibody can, in certain instances, comprise two identical antigen-binding sites, each of which specifically binds the same epitope on two or more proteins. In certain alternative embodiments, an antibody can be bispecific and comprise at least two antigen-binding sites with differing specificities. Generally, but not necessarily, reference to “binding” means “specific binding”.

“Binding affinity” generally refers to the strength of the sum total of noncovalent interactions between a single binding site of a molecule (e.g., a binding agent such as an antibody) and its binding partner (e.g., an antigen such as NKG2A). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. In one embodiment, the “K_{D}” or “K_{D} value” may be measured by biolayer interferometry (BLI) using, for example, the OctetQK384 system (ForteBio, Menlo Park, CA). Alternatively, the K_{D} may also be measured in a radiolabeled antigen-binding assay (RIA), for example, performed with the Fab version of an antibody of interest and its antigen (Chen, et al., (1999) J. Mol Biol 293:865-881) or using surface plasmon resonance (SPR) assays by BIACORE™, using, for example, a BIACORE™-2000 or a BIACORE™-3000 (BIACORE™, Inc., Piscataway, NJ). An “on-rate” or “rate of association” or “association rate” or “kₒₙ” as well as an “off-rate” or “rate of dissociation” or “dissociation rate” or “k_{off}” can also be determined with the same SPR or BLI techniques described above using, for example, the OctetQK384 system (ForteBio, Menlo Park, CA) or a BIACORE™-2000 or a BIACORE™-3000 (BIACORE™, Inc., Piscataway, NJ), respectively.

As used herein, and unless otherwise specified, the term “subject” or “patient” refers to an animal, including, but not limited to, a mammal, including a primate (e.g., human), cow, sheep, goat, horse, dog, cat, rabbit, rat, or mouse. The terms “subject” and “patient” are used interchangeably herein in reference, for example, to a mammalian subject, such as a human subject.

As used herein, and unless otherwise specified, the terms “treat”, “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a patient with such a disease or disorder. In some embodiments, the terms refer to the administration of the compounds provided herein, with or without other additional active agent, after the onset of symptoms of the particular disease.

As used herein, and unless otherwise specified, the terms “prevent”, “preventing” and “prevention” refer to the prevention of the onset, recurrence or spread of a disease or disorder, or of one or more symptoms thereof. In certain embodiments, the terms refer to the treatment with or administration of the compounds provided herein, with or without other additional active compound, prior to the onset of symptoms, particularly to patients at risk of diseases or disorders provided herein. The terms encompass the inhibition or reduction of a symptom of the particular disease. Patients with familial history of a disease in particular are candidates for preventive regimens in certain embodiments. In addition, patients who have a history of recurring symptoms are also potential candidates for the prevention. In this regard, the term “prevention” may be interchangeably used with the term “prophylactic treatment”.

As used herein, and unless otherwise specified, the terms “manage”, “managing” and “management” refer to preventing or slowing the progression, spread or worsening of a disease or disorder, or of one or more symptoms thereof. Often, the beneficial effects that a patient derives from a prophylactic and/or therapeutic agent do not result in a cure of the disease or disorder. In this regard, the term “managing” encompasses treating a patient who had suffered from the particular disease in an attempt to prevent or minimize the recurrence of the disease, or lengthening the time during which the disease remains in remission.

As used herein, and unless otherwise specified, a “therapeutically effective amount” of a compound is an amount sufficient to provide a therapeutic benefit in the treatment or management of a disease or disorder, or to delay or minimize one or more symptoms associated with the disease or disorder. A “therapeutically effective amount” of a compound means an amount of therapeutic agent, alone or in combination with other therapies, which provides a therapeutic benefit in the treatment or management of the disease or disorder. The term “therapeutically effective amount” can encompass an amount that improves overall therapy, reduces or avoids symptoms or causes of disease or disorder, or enhances the therapeutic efficacy of another therapeutic agent.

As used herein, and unless otherwise specified, a “prophylactically effective amount” of a compound is an amount sufficient to prevent a disease or disorder, or prevent its recurrence. A “prophylactically effective amount” of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

“Combination therapy” or “in combination with” refer to the use of more than one therapeutic agent to treat a particular disorder or condition. By “in combination with” it is not intended to imply that the therapeutic agents must be administered at the same time and/or formulated for delivery together, although these methods of delivery are within the scope of this disclosure. A therapeutic agent can be administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), one or more other additional agents. The therapeutic agents in a combination therapy can also be administered on an alternating dosing schedule, with or without a resting period (e.g., no therapeutic agent is administered on certain days of the schedule). The administration of a therapeutic agent “in combination with” another therapeutic agent includes, but is not limited to, sequential administration and concomitant administration of the two agents. In general, each therapeutic agent is administered at a dose and/or on a time schedule determined for that particular agent.

As used herein, and unless otherwise specified, the term “pharmaceutically acceptable carrier”, “pharmaceutically acceptable excipient”, “physiologically acceptable carrier”, or “physiologically acceptable excipient” refers to a pharmaceutically-acceptable material, composition, or vehicle, such as a liquid or solid filler, diluent, excipient, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. (See, Remington: The Science and Practice of Pharmacy, 21st Edition, Lippincott Williams & Wilkins: Philadelphia, PA, 2005; Handbook of Pharmaceutical Excipients, 5th Edition, Rowe et al., Eds., The Pharmaceutical Press and the American Pharmaceutical Association: 2005; Handbook of Pharmaceutical Additives, 3rd Edition, Ash and Ash Eds., Gower Publishing Company: 2007; and Pharmaceutical Preformulation and Formulation, Gibson Ed., CRC Press LLC: Boca Raton, FL, 2004).

As used herein, and unless otherwise specified, the term “relapsed” refers to a situation where a subject or a mammal, which has had a remission of cancer after therapy has a return of cancer cells. As used herein, and unless otherwise specified, the term “refractory” or “resistant” refers to a circumstance where a subject or a mammal, even after intensive treatment, has residual cancer cells in his/her body.

In one embodiment, “relapsed” DLBCL may refer to DLBCL that has been previously treated with one or more lines of therapy. In one embodiment, the relapsed DLBCL is DLBCL that has been previously treated with one, two, three or four lines of therapy. In one embodiment, the relapsed DLBCL is DLBCL that has been previously treated with two or more lines of treatment.

In one embodiment, “relapsed” FL may refer to FL that has been previously treated with one or more lines of therapy. In one embodiment, the relapsed FL is FL that has been previously treated with one, two, three or four lines of therapy. In one embodiment, the relapsed FL is FL that has been previously treated with two or more lines of treatment.

In one embodiment, a relapsed or refractory DLBCL has been previously treated with at least one prior line of therapy. In one embodiment, a relapsed or refractory DLBCL has been previously treated with at least two prior lines of therapy. In one embodiment, no more than one of the prior lines of therapy may be a treatment for lower grade lymphoma. In one embodiment, a relapsed or refractory DLBCL has been previously treated with at least one standard treatment regimen for DLBCL. In one embodiment, a relapsed or refractory DLBCL has been previously treated with one or more systemic regimens, wherein one or more of the systemic regimens comprise an anti-CD20 therapy. In certain embodiments, the subject has received one to three systemic regimens, wherein at least one of the systemic regimens is an anti-CD20 therapy.

In one embodiment, a relapsed or refractory FL has been previously treated with at least one prior line of therapy. In one embodiment, a relapsed or refractory FL has been previously treated with at least two prior lines of therapy. In one embodiment, a relapsed or refractory FL is Grade 1, 2, 3a or 3b according to Groupe d'Etude des Lymphomes Folliculaires (GELF) criteria (National Comprehensive Cancer Network. NCCN Clinical Practice Guidelines in Oncology: B-cell Lymphomas; V.2.2018. 2018 Feb. 26; V.2, the entirety of which is incorporated herein by reference). In one embodiment, a relapsed or refractory FL has been previously treated with one or more systemic regimens, wherein one or more of the systemic regimens comprise an anti-CD20 therapy. In certain embodiments, the subject has received one to three systemic regimens, wherein at least one of the systemic regimens is an anti-CD20 therapy.

As used herein, “induction therapy” refers to the first treatment given for a disease, or the first treatment given with the intent of inducing complete remission in a disease, such as cancer. When used by itself, induction therapy is the one accepted as the best available treatment. If residual cancer is detected, patients are treated with another therapy, termed reinduction. If the patient is in complete remission after induction therapy, then additional consolidation and/or maintenance therapy is given to prolong remission or to potentially cure the patient.

As used herein, “consolidation therapy” refers to the treatment given for a disease after remission is first achieved. For example, consolidation therapy for cancer is the treatment given after the cancer has disappeared after initial therapy. Consolidation therapy may include radiation therapy, stem cell transplant, or treatment with cancer drug therapy. Consolidation therapy is also referred to as intensification therapy and post-remission therapy.

As used herein, “maintenance therapy” refers to the treatment given for a disease after remission or best response is achieved, in order to prevent or delay relapse. Maintenance therapy can include chemotherapy, hormone therapy or targeted therapy.

As used herein, and unless otherwise specified, an “effective patient tumor response” refers to any increase in the therapeutic benefit to the patient. An “effective patient tumor response” can be, for example, a 5%, 10%, 25%, 50%, or 100% decrease in the rate of progress of the tumor. An “effective patient tumor response” can be, for example, a 5%, 10%, 25%, 50%, or 100% decrease in the physical symptoms of a cancer. An “effective patient tumor response” can also be, for example, a 5%, 10%, 25%, 50%, 100%, 200%, or more increase in the response of the patient, as measured by any suitable means, such as gene expression, cell counts, assay results, etc.

An improvement in the cancer or cancer-related disease can be characterized as a complete or partial response. “Complete response” refers to an absence of clinically detectable disease with normalization of any previously abnormal radiographic studies, bone marrow, and cerebrospinal fluid (CSF) or abnormal monoclonal protein measurements. “Partial response” refers to at least about a 10%, 20%, 30%, 40%, 50%, 60%, 70%, 80%, or 90% decrease in all measurable tumor burden (i.e., the number of malignant cells present in the subject, or the measured bulk of tumor masses or the quantity of abnormal monoclonal protein) in the absence of new lesions. The term “treatment” contemplates both a complete and a partial response.

As used herein, and unless otherwise specified, the term “likelihood” generally refers to an increase in the probability of an event. The term “likelihood” when used in reference to the effectiveness of a patient tumor response generally contemplates an increased probability that the rate of tumor progress or tumor cell growth will decrease. The term “likelihood” when used in reference to the effectiveness of a patient tumor response can also generally mean the increase of indicators, such as mRNA or protein expression, that may evidence an increase in the progress in treating the tumor.

As used herein, and unless otherwise specified, the term “predict” generally means to determine or tell in advance. When used to “predict” the effectiveness of a cancer treatment, for example, the term “predict” can mean that the likelihood of the outcome of the cancer treatment can be determined at the outset, before the treatment has begun, or before the treatment period has progressed substantially.

As used herein, and unless otherwise specified, the term “monitor” generally refers to the overseeing, supervision, regulation, watching, tracking, or surveillance of an activity. For example, the term “monitoring the effectiveness of a compound” refers to tracking the effectiveness in treating a cancer in a patient or in a tumor cell culture. Similarly, the “monitoring”, when used in connection with patient compliance, either individually, or in a clinical trial, refers to the tracking or confirming that the patient is actually taking the compound being tested as prescribed. The monitoring can be performed, for example, by following the expression of mRNA or protein biomarkers.

As used herein, and unless otherwise specified, the term “drug resistance” refers to the condition when a disease does not respond to the treatment of a drug or drugs. Drug resistance can be either intrinsic, which means the disease has never been responsive to the drug or drugs, or it can be acquired, which means the disease ceases responding to a drug or drugs that the disease had previously responded to. In certain embodiments, drug resistance is intrinsic. In certain embodiments, drug resistance is acquired.

As used herein, and unless otherwise specified, the term “sensitivity” and “sensitive” when made in reference to treatment with a compound is a relative term which refers to the degree of effectiveness of the compound in lessening or decreasing the progress of a tumor or the disease being treated. For example, the term “increased sensitivity” when used in reference to treatment of a cell or tumor in connection with a compound refers to an increase, such as an increase of at least 5%, or more, in the effectiveness of the tumor treatment.

As used herein, and unless otherwise specified, the terms “determining”, “measuring”, “evaluating”, “assessing” and “assaying” as used herein generally refer to any form of measurement, and include determining if an element is present or not. These terms include both quantitative and/or qualitative determinations. Assessing may be relative or absolute. “Assessing the presence of” can include determining the amount of something present, as well as determining whether it is present or absent.

### Clinical Study Endpoints

In the context of a cancer, inhibition may be assessed by inhibition of disease progression, inhibition of tumor growth, reduction of primary tumor, relief of tumor-related symptoms, inhibition of tumor secreted factors, delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), increased Overall Survival (OS), among others. OS as used herein means the time from treatment onset until death from any cause. TTP as used herein means the time from treatment onset until tumor progression; TTP does not include deaths. In one embodiment, PFS means the time from treatment onset until tumor progression or death. In one embodiment, PFS means the time from the first dose of compound to the first occurrence of disease progression or death from any cause. In one embodiment, PFS rates will be computed using the Kaplan-Meier estimates. Event-free survival (EFS) means the time from treatment onset until any treatment failure, including disease progression, treatment discontinuation for any reason, or death. In one embodiment, overall response rate (ORR) means the percentage of patients who achieve a response. In one embodiment, ORR means the sum of the percentage of patients who achieve complete and partial responses. In one embodiment, ORR means the percentage of patients whose best response ≥partial response (PR), according to the IMWG Uniform Response Criteria. In one embodiment, duration of response (DoR) is the time from achieving a response until relapse or disease progression. In one embodiment, DoR is the time from achieving a response ≥partial response (PR) until relapse or disease progression. In one embodiment, DoR is the time from the first documentation of a response until the first documentation of progressive disease or death. In one embodiment, DoR is the time from the first documentation of a response ≥partial response (PR) until the first documentation of progressive disease or death. In one embodiment, time to response (TTR) means the time from the first dose of compound to the first documentation of a response. In one embodiment, TTR means the time from the first dose of compound to the first documentation of a response ≥partial response (PR). In the extreme, complete inhibition, is referred to herein as prevention or chemoprevention. In this context, the term “prevention” includes either preventing the onset of clinically evident cancer altogether or preventing the onset of a preclinically evident stage of a cancer. Also intended to be encompassed by this definition is the prevention of transformation into malignant cells or to arrest or reverse the progression of premalignant cells to malignant cells. This includes prophylactic treatment of those at risk of developing a cancer.

In certain embodiments, the treatment of NHL may be assessed by the International Workshop Criteria for Malignant Lymphoma (see Cheson et al., J. Clin. Oncol., 2014, 32(27):3059-3068; the entirety of which is incorporated herein by reference) and the Deauville Criteria for fluorodeoxyglucose-positron emission tomography (FDG-PET) scan interpretation (Itti et al., Eur. J. Nucl. Med. Mol. Imaging, 2013, 40(9):1312-20; Meignan et al., Leuk Lymphoma, 2014, 55(1):31-37; the entirety of each of which is incorporated herein by reference) (“Lugano criteria”), using the response and end point definition shown in Tables 4-6.

TABLE 4
Criteria for Involvement of Site.

| Tissue Site | Clinical | FDG Avidity | Test | Positive Finding |
|---|---|---|---|---|
| Lymph nodes | Palpable | FDG-avid histologies | PET/CT | Increase FDG uptake |
| | | Nonavid disease | CT | Unexplained node |
| | | | | enlargement |
| Spleen | Palpable | FDG-avid histologies | PET/CT | Diffuse uptake, solitary |
| | | | | mass, miliary lesions, |
| | | | | nodules |
| | | Nonavid disease | CT | >13 cm in vertical length, |
| | | | | mass, nodules |
| Liver | Palpable | FDG-avid histologies | PET/CT | Diffuse uptake, mass |
| | | Nonavid disease | CT | Nodules |
| CNS | Signs, | N/A | CT | Mass lesion(s) |
| | symptoms | | MRI | Leptomeningeal |
| | | | | infiltration, mass lesions |
| | | | CSF | Cytology, flow cytometry |
| | | | assessment | |
| Other (e.g., skin, | Site dependent | N/A | PET/CT ^{a}, | Lymphoma involvement |
| lung, GI tract, | | | biopsy | |
| bone, bone | | | | |
| marrow) | | | | |

CNS = central nervous system; CSF = cerebrospinal fluid; CT = computed tomography; FDG = fluorodeoxyglucose; GI = gastrointestinal; MRI = magnetic resonance imaging; PET = positron emission tomography; N/A = not applicable.
^{a} PET/CT is adequate for determination of bone marrow involvement and can be considered highly suggestive for involvement of other extralymphatic sites. Biopsy confirmation of those sites can be considered if necessary.

TABLE 5
Lugano Response Criteria for Non-Hodgkin Lymphoma.

| Response | Site | PET/CT (metabolic response) | CT (Radiologic response) |
|---|---|---|---|
| Complete | Lymph nodes | Score 1, 2, 3 with or without | All of the following: |
| response | and | residual mass on 5-PS (Table 6) | Target nodes/nodal masses must |
| | extralymphatic | | regress to ≤1.5 cm in LDi |
| | sites | | No extralymphatic sites of disease |
| | Non-measured | N/A | Absent |
| | lesion | | |
| | Organ | N/A | Regress to normal |
| | enlargement | | |
| | New Lesions | None | None |
| | Bone Marrow | No evidence of FDG-avid disease | Normal by morphology; if indeterminate, |
| | | in marrow | IHC negative ^{a} |
| Partial | Lymph nodes | Score 4 or 5 on 5-PS with reduced | All of the following: |
| Response | and | uptake compared with baseline and | ≥50% decrease in SPD of up to 6 target |
| | extralymphatic | residual mass(es) of any size. | measurable nodes and extranodal sites |
| | sites | At interim these findings suggest | When a lesion is too small to measure on |
| | | responding disease. | CT, assign 5 mm × 5 mm as the default |
| | | At end of treatment these findings | value |
| | | may indicate residual disease. | When no longer visible, 0 mm × 0 mm |
| | | | For a node >5 mm × 5 mm, but smaller |
| | | | than normal, use actual measurement for |
| | | | calculation |
| | Non-measured | N/A | Absent/normal, regressed, but no increase |
| | lesion | | |
| | Organ | N/A | Spleen must have regressed by >50% in |
| | enlargement | | length beyond normal |
| | New Lesions | None | None |
| | Bone Marrow | Residual uptake higher than uptake | N/A |
| | | in normal marrow but reduced | |
| | | compared with baseline. If | |
| | | persistent focal changes in the | |
| | | marrow in the context of nodal | |
| | | response, consider MRI or biopsy | |
| | | or interval scan. | |
| Stable | Target | Score 4 or 5 on 5-PS with no | <50% decrease from baseline of up to 6 |
| Disease | nodes/nodal | significant change in FDG uptake | dominant, measurable nodes and |
| | masses, | from baseline | extranodal sites |
| | extranodal | | No criteria for progressive disease are met |
| | lesions | | |
| | Non-measured | N/A | No increase consistent with progression |
| | lesion | | |
| | Organ | N/A | No increase consistent with progression |
| | enlargement | | |
| | New Lesions | None | None |
| | Bone Marrow | No change from baseline | N/A |
| Progressive | Lymph nodes | Score 4 or 5 on 5-PS with an | At least one of the following: |
| Disease | and | increase in intensity of uptake | PPD progression: |
| | extralymphatic | compared with baseline | An individual node/lesion must be |
| | sites | and/or | abnormal with: |
| | | New FDG-avid foci consistent with | LDi >1.5 cm and |
| | | lymphoma | Increase by ≥50% from PPD nadir and |
| | | | An increase in LDi or SDi from nadir |
| | | | 0.5 cm for lesions ≤2 cm |
| | | | 1.0 cm for lesions >2 cm |
| | | | In the setting of splenomegaly, splenic |
| | | | length must increase by >50% of the |
| | | | extent of its prior increase above baseline |
| | | | (e.g., a 15 cm spleen must increase to >16 |
| | | | cm). If no splenomegaly, must increase by |
| | | | at least 2 cm from baseline |
| | | | New or recurrent splenomegaly |
| | Non-measured | None | New or clear progression of preexisting |
| | lesion | | non-measured lesions |
| | New Lesions | New FDG-avid foci consistent with | Regrowth of previously resolved lesions |
| | | lymphoma rather than another | A new node >1.5 cm in any axis |
| | | etiology (e.g., infection, | A new extranodal site >1.0 cm in any axis; |
| | | inflammation). If uncertain | if <1.0 cm in any axis, its presence must |
| | | etiology, consider biopsy or | be unequivocal and must be attributable to |
| | | interval scan. | lymphoma |
| | | | Assessable disease of any size |
| | | | unequivocally attributable to lymphoma |
| | Bone Marrow | New or recurrent FDG-avid foci | New or recurrent involvement |

CMR = complete metabolic response; LDi = longest transverse diameter of a lesion; PPD = cross product of the LDi and perpendicular diameter; SDi = shortest axis perpendicular to the LDi; SPD = sum of the product of the perpendicular diameters for multiple lesions; N/A = not applicable.
^{a} Required for CR if bone marrow involvement at baseline
^{b} In Waldeyer's ring or extranodal sites with high physiologic uptake or with activation within spleen or marrow (e.g. with chemotherapy or myeloid colony stimulating factors), uptake may be greater than normal mediastinum and/or liver. In this circumstance, CMR may be inferred if uptake at sites of initial involvement is no greater than surrounding normal tissue.
^{c} FDG-avid lymphomas should have response assessed by PET-CT. Some diseases can typically be followed with CT alone (i.e., marginal zone lymphoma).
^{d} PET should be done with contrast-enhanced diagnostic CT and can be done simultaneously or at separate procedures.

TABLE 6
PET Five Point Scale (5-PS).

| | |
|---|---|
| 1 | No uptake above background |
| 2 | Uptake ≤ mediastinum |
| 3 | Uptake > mediastinum but ≤ liver |
| 4 | Uptake moderately > liver |
| 5 | Uptake markedly higher than liver and/or new lesions |
| X | New areas of uptake unlikely to be related to lymphoma |

^{a} The Deauville five-point scale (5-PS) is an internationally recommended scale for clinical routine and clinical trials using FDG-PET/CT in the initial staging and assessment of treatment response in Hodgkin lymphoma (HL) and certain types of non-Hodgkin lymphomas (NHL).

In certain embodiments, stable disease or lack thereof can be determined by methods known in the art such as evaluation of patient symptoms, physical examination, visualization of the tumor that has been imaged, for example using FDG-PET (fluorodeoxyglucose positron emission tomography), PET/CT (positron emission tomography/computed tomography), MRI (magnetic resonance imaging) of the brain/spine, examination of the CSF (cerebrospinal fluid), ophthalmologic exams, vitreal fluid sampling, retinal photograph, bone marrow evaluation and other commonly accepted evaluation modalities.

In certain embodiments, the methods provided herein are useful for achieving one or more of these clinical trial endpoints in a patient. In certain embodiments, the methods provided herein are useful for improving one or more of these clinical trial endpoints in a patient.

### COMPOUND

In certain embodiments, the compound for use in the compositions and methods provided herein is 2-(2,6-dioxopiperidin-3-yl)-4-((2-fluoro-4-((3-morpholinoazetidin-1-yl)methyl)benzyl)amino)isoindoline-1,3-dione (Compound A), having the following structure:

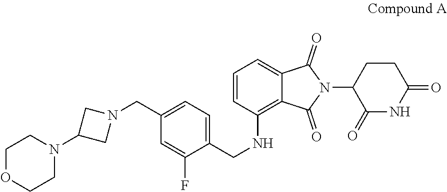 [SMILES: O=C1CCC(N2C(=O)c3cccc(NCc4ccc(CN5CC(N6CCOCC6)C5)cc4F)c3C2=O)C(=O)N1]

or an enantiomer or mixture of enantiomers, a tautomer, an isotopolog, a pharmaceutically acceptable salt, solvate, hydrate, co-crystal, clathrate, or polymorph thereof.

In one embodiment, the compound is 2-(2,6-dioxopiperidin-3-yl)-4-((2-fluoro-4-((3-morpholinoazetidin-1-yl)methyl)benzyl)amino)isoindoline-1,3-dione. In one embodiment, the compound is a pharmaceutically acceptable salt of Compound A. In one embodiment, the compound is 2-(2,6-dioxopiperidin-3-yl)-4-((2-fluoro-4-((3-morpholinoazetidin-1-yl)methyl)benzyl)amino)isoindoline-1,3-dione hydrochloride.

In one embodiment, the compound is (S)-2-(2,6-dioxopiperidin-3-yl)-4-((2-fluoro-4-((3-morpholinoazetidin-1-yl)methyl)benzyl)amino)isoindoline-1,3-dione (Compound A-S), having the following structure:

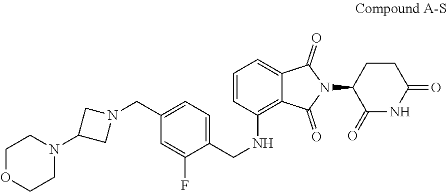 [SMILES: O=C1CC[C@H](N2C(=O)c3cccc(NCc4ccc(CN5CC(N6CCOCC6)C5)cc4F)c3C2=O)C(=O)N1]

In one embodiment, the compound is a pharmaceutically acceptable salt of Compound A-S. In one embodiment, the compound is a hydrochloride salt of Compound A-S. Certain salts and polymorphic forms of Compound A-S are described in U.S. Patent Publication No. 2021/0115019 A1, the entirety of which is incorporated herein by reference.

In one embodiment, the compound is (R)-2-(2,6-dioxopiperidin-3-yl)-4-((2-fluoro-4-((3-morpholinoazetidin-1-yl)methyl)benzyl)amino)isoindoline-1,3-dione (Compound A-R), having the following structure:

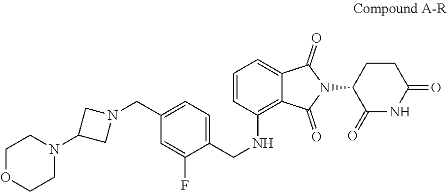 [SMILES: O=C1CC[C@@H](N2C(=O)c3cccc(NCc4ccc(CN5CC(N6CCOCC6)C5)cc4F)c3C2=O)C(=O)N1]

In one embodiment, the compound is a pharmaceutically acceptable salt of Compound A-R. In one embodiment, the compound is a hydrochloride salt of Compound A-R.

Compound A, A-S, or A-R can be prepared according to the methods described in U.S. Patent Publication Nos. 2019/0322647 A1, 2020/0325129 A1, 2021/0113574 A1, 2021/0113576 A1, and 2021/0113577 A1, the entirety of each of which is incorporated herein by reference. The compound can be also synthesized according to other methods apparent to those of skill in the art based upon the teaching of these publications.

Compounds provided herein markedly inhibit TNF-α, IL-1β, and other inflammatory cytokines in LPS-stimulated hPBMC and human whole blood. TNF-α is an inflammatory cytokine produced by macrophages and monocytes during acute inflammation. TNF-α is responsible for a diverse range of signaling events within cells. TNF-α may play a pathological role in cancer. Without being limited by theory, one of the biological effects exerted by the immunomodulatory compounds provided herein is the reduction of synthesis of TNF-α. The immunomodulatory compounds provided herein enhance the degradation of TNF-α mRNA. The compounds provided herein also potently inhibit IL-1β and stimulate IL-1β under these conditions.

Further, without being limited by any particular theory, the compounds provided herein are potent co-stimulators of T-cells and increase cell proliferation in a dose dependent manner under appropriate conditions.

In certain embodiments, without being limited by theory, the biological effects exerted by the immunomodulatory compounds provided herein include, but are not limited to, anti-angiogenic and immune modulating effects.

Compound A provided herein contains one chiral center, and can exist as a mixture of enantiomers, e.g., a racemic mixture. This disclosure encompasses the use of stereomerically pure forms of such a compound, as well as the use of mixtures of those forms. For example, mixtures comprising equal or unequal amounts of the enantiomers of Compound A provided herein may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN, 1972).

### Bispecific Antibody

Provided herein are compositions comprising, and methods using, the bispecific antibody glofitamab or the bispecific antibody mosunetuzumab.

Provided herein are compositions comprising, and methods using, the bispecific antibody glofitamab. Glofitamab is an anti-CD20/anti-CD3 bispecific antibody (WHO Drug Information (International Nonproprietary Names for Pharmaceutical Substances), Recommended INN: List 83, 2020, vol. 34, no. 1, p. 39; Proposed INN: List 121 WHO Drug Information, vol. 33, no. 2, 2019, page 276, also known as CD20-TCB, RO7082859, or RG6026; CAS #: 2229047-91-8). Glofitamab is a novel T-cell-engaging bispecific (TCB) full-length antibody with a 2:1 molecular configuration for bivalent binding to CD20 on B-cells and monovalent binding to CD3, particularly the CD3 epsilon chain (CD3ε), on T-cells. Its CD3-binding region is fused to one of the CD20-binding regions in a head-to-tail fashion via a flexible linker. This structure endows glofitamab with superior in vitro potency versus other CD20-CD3 bispecific antibodies with a 1:1 configuration and leads to profound antitumor efficacy in preclinical DLBCL models. CD20 bivalency preserves this potency in the presence of competing anti-CD20 antibodies, providing the opportunity for pre- or co-treatment with these agents. Glofitamab comprises an engineered, heterodimeric Fc region with completely abolished binding to FcgRs and C1q. By simultaneously binding to human CD20-expressing tumor cells and to the CD3ε of the T-cell receptor (TCR) complex on T-cells, it induces tumor cell lysis, in addition to T-cell activation, proliferation and cytokine release. Lysis of B-cells mediated by glofitamab is CD20-specific and does not occur in the absence of CD20 expression or in the absence of simultaneous binding (cross-linking) of T-cells to CD20-expressing cells. In addition to killing, T-cells undergo activation due to CD3 cross-linking, as detected by an increase in T-cell activation markers (CD25 and CD69), cytokine release (IFNγ, TNFα, IL-2, IL-6, and IL-10), cytotoxic granule release (Granzyme B) and T-cell proliferation. The sequences of glofitamab are summarized in Table 1 above.

Provided herein are compositions comprising, and methods using, the bispecific antibody mosunetuzumab. Mosunetuzumab has the International Nonproprietary Names for Pharmaceutical Substances (INN) List 117 (WHO Drug Information, vol. 31, no. 2, 2017, p. 303), or CAS Registry No. 1905409-39-3, and has (1) an anti-CD20 arm comprising the heavy chain and light chain sequences of SEQ ID NOs: 22 and 23, respectively, and (2) an anti-CD3 arm comprising the heavy chain and light chain sequences of SEQ ID NOs: 24 and 25, respectively. Amino acid sequences of mosunetuzumab are summarized in Table 2 above.

Mosunetuzumab may be produced using recombinant methods and compositions, for example, as described in U.S. Pat. No. 4,816,567, the entirety of which is incorporated herein by reference. More generally, antibodies can be made by culturing a host cell comprising a nucleic acid encoding the antibody, under conditions suitable for expression of the antibody, and recovering the antibody from the host cell (or host cell culture medium). Glofitamab is provided under the trade name Columvi®. Mosunetuzumab is provided under the trade name Lunsumio®.

### Methods of Treatment and Composition for Use in Such Methods

The compounds provided herein in combination with the bispecific antibody glofitamab or in combination with the bispecific antibody mosunetuzumab can be used in all methods of treatment as provided herein.

In certain embodiments, provided herein is a method of treating or managing non-Hodgkin lymphoma, comprising administering to a subject having non-Hodgkin lymphoma (a) a therapeutically effective amount of a compound provided herein in combination with (b) a therapeutically effective amount of a bispecific antibody, wherein the bispecific antibody is glofitamab or mosunetuzumab.

### Method of Treatment

In certain embodiments, provided herein is a method of treating or managing non-Hodgkin lymphoma (NHL), comprising administering to a subject having the NHL:
(a) a therapeutically effective amount of a compound, wherein the compound is Compound A, A-S, or A-R

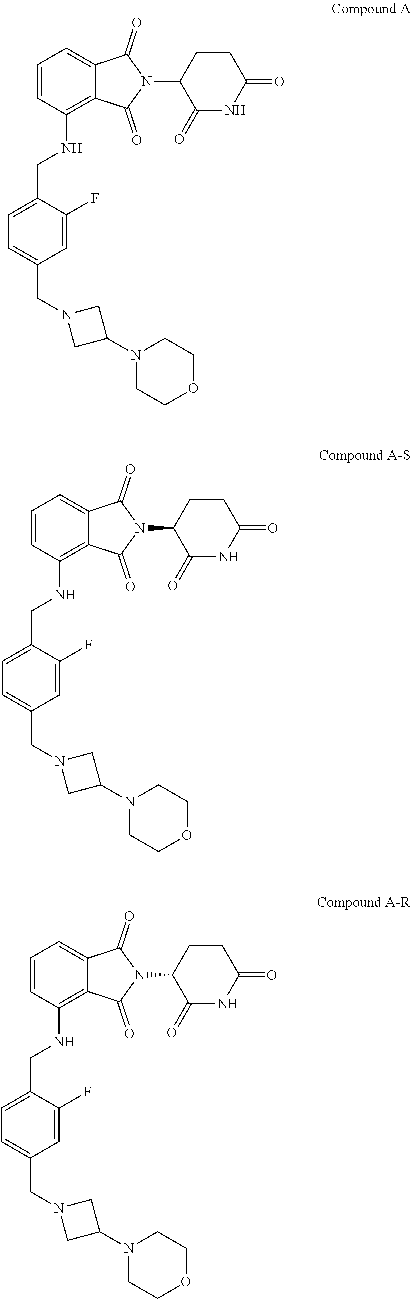 [SMILES: O=C1CCC(N2C(=O)c3cccc(NCc4ccc(CN5CC(N6CCOCC6)C5)cc4F)c3C2=O)C(=O)N1 ; O=C1CC[C@H](N2C(=O)c3cccc(NCc4ccc(CN5CC(N6CCOCC6)C5)cc4F)c3C2=O)C(=O)N1 ; O=C1CC[C@@H](N2C(=O)c3cccc(NCc4ccc(CN5CC(N6CCOCC6)C5)cc4F)c3C2=O)C(=O)N1]

or an enantiomer or mixture of enantiomers, a tautomer, an isotopolog, a pharmaceutically acceptable salt, solvate, hydrate, co-crystal, clathrate, or polymorph thereof, in combination with
(b) a therapeutically effective amount of a bispecific antibody, wherein the bispecific antibody is glofitamab or mosunetuzumab.

In certain embodiments, provided herein is a method of treating or managing non-Hodgkin lymphoma (NHL), comprising administering to a subject having the NHL: (a) a therapeutically effective amount of a compound provided herein in combination with (b) a therapeutically effective amount of a bispecific antibody, wherein the bispecific antibody is glofitamab or mosunetuzumab.

### Compound for Use

In certain embodiments, provided herein is a compound for use in a method of treating non-Hodgkin lymphoma (NHL), wherein the method comprises administering to a subject having the NHL:
(a) a therapeutically effective amount of the compound, wherein the compound is Compound A, A-S, or A-R

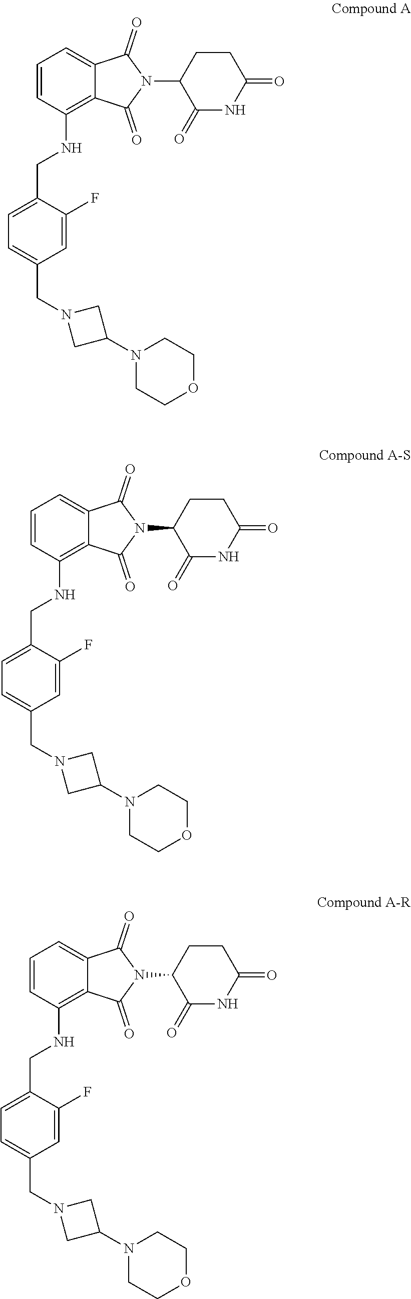 [SMILES: O=C1CCC(N2C(=O)c3cccc(NCc4ccc(CN5CC(N6CCOCC6)C5)cc4F)c3C2=O)C(=O)N1 ; O=C1CC[C@H](N2C(=O)c3cccc(NCc4ccc(CN5CC(N6CCOCC6)C5)cc4F)c3C2=O)C(=O)N1 ; O=C1CC[C@@H](N2C(=O)c3cccc(NCc4ccc(CN5CC(N6CCOCC6)C5)cc4F)c3C2=O)C(=O)N1]

or an enantiomer or mixture of enantiomers, a tautomer, an isotopolog, a pharmaceutically acceptable salt, solvate, hydrate, co-crystal, clathrate, or polymorph thereof, in combination with
(b) a therapeutically effective amount of a bispecific antibody, wherein the bispecific antibody is glofitamab or mosunetuzumab.

In certain embodiments, provided herein is a compound for use in a method of treating non-Hodgkin lymphoma (NHL), wherein the method comprises administering to a subject having the NHL: (a) a therapeutically effective amount of the compound provided herein in combination with (b) a therapeutically effective amount of a bispecific antibody, wherein the bispecific antibody is glofitamab or mosunetuzumab.

### Bispecific Antibody for Use

In certain embodiments, provided herein is a bispecific antibody for use in a method of treating non-Hodgkin lymphoma (NHL), wherein the method comprises administering to a subject having the NHL:
(a) a therapeutically effective amount of a compound, wherein the compound is Compound A, A-S, or A-R

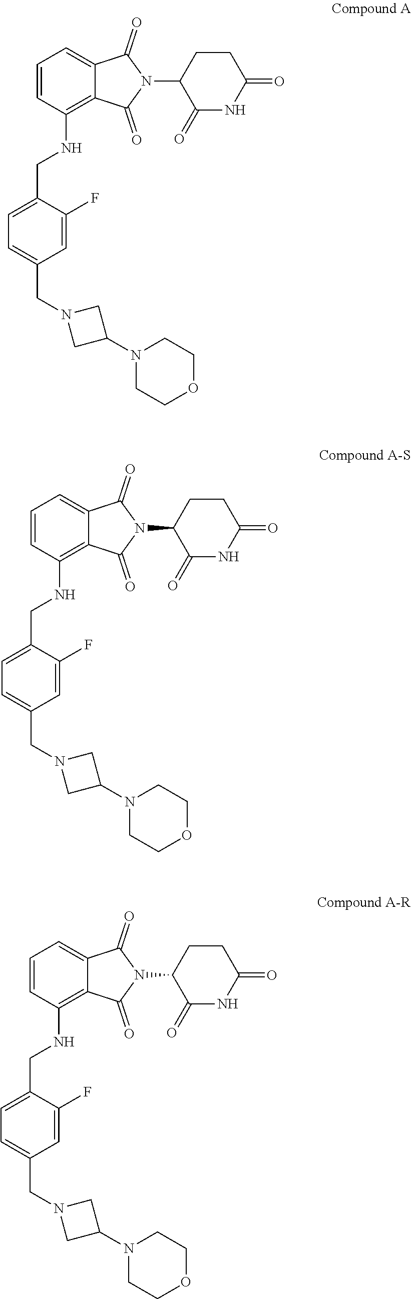 [SMILES: O=C1CCC(N2C(=O)c3cccc(NCc4ccc(CN5CC(N6CCOCC6)C5)cc4F)c3C2=O)C(=O)N1 ; O=C1CC[C@H](N2C(=O)c3cccc(NCc4ccc(CN5CC(N6CCOCC6)C5)cc4F)c3C2=O)C(=O)N1 ; O=C1CC[C@@H](N2C(=O)c3cccc(NCc4ccc(CN5CC(N6CCOCC6)C5)cc4F)c3C2=O)C(=O)N1]

or an enantiomer or mixture of enantiomers, a tautomer, an isotopolog, a pharmaceutically acceptable salt, solvate, hydrate, co-crystal, clathrate, or polymorph thereof, in combination with
(b) a therapeutically effective amount of the bispecific antibody, wherein the bispecific antibody is glofitamab or mosunetuzumab.

In certain embodiments, provided herein is a bispecific antibody for use in a method of treating non-Hodgkin lymphoma (NHL), wherein the method comprises administering to a subject having the NHL: (a) a therapeutically effective amount of a compound provided herein in combination with (b) a therapeutically effective amount of the bispecific antibody, wherein the bispecific antibody is glofitamab or mosunetuzumab.

### Compound and Bispecific Antibody for Use

In certain embodiments, provided herein is a compound and a bispecific antibody for use in a method of treating non-Hodgkin lymphoma (NHL), wherein the method comprises administering to a subject having the NHL:
(a) a therapeutically effective amount of the compound, wherein the compound is Compound A, A-S, or A-R

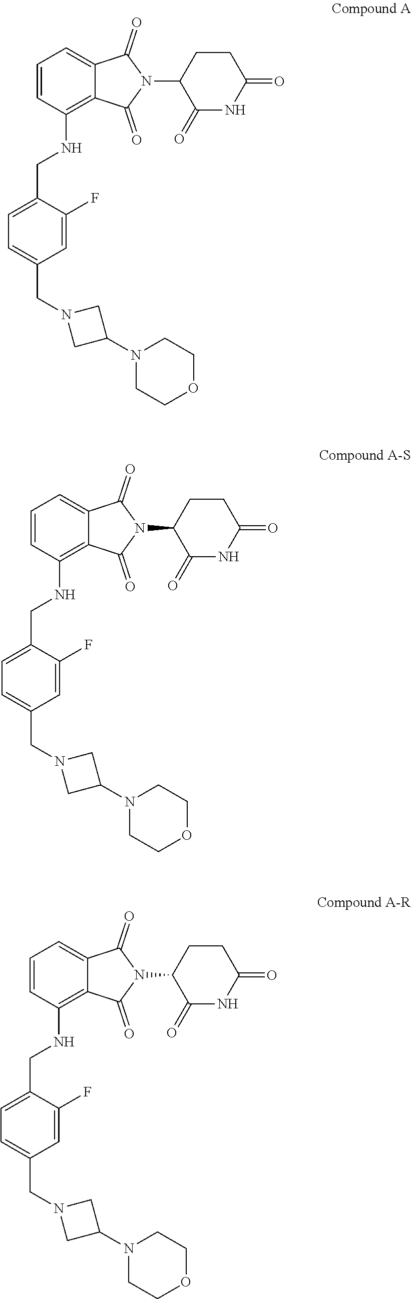 [SMILES: O=C1CCC(N2C(=O)c3cccc(NCc4ccc(CN5CC(N6CCOCC6)C5)cc4F)c3C2=O)C(=O)N1 ; O=C1CC[C@H](N2C(=O)c3cccc(NCc4ccc(CN5CC(N6CCOCC6)C5)cc4F)c3C2=O)C(=O)N1 ; O=C1CC[C@@H](N2C(=O)c3cccc(NCc4ccc(CN5CC(N6CCOCC6)C5)cc4F)c3C2=O)C(=O)N1]

or an enantiomer or mixture of enantiomers, a tautomer, an isotopolog, a pharmaceutically acceptable salt, solvate, hydrate, co-crystal, clathrate, or polymorph thereof, in combination with
(b) a therapeutically effective amount of the bispecific antibody, wherein the bispecific antibody is glofitamab or mosunetuzumab.

In certain embodiments, provided herein is a compound and a bispecific antibody for use in a method of treating non-Hodgkin lymphoma (NHL), wherein the method comprises administering to a subject having the NHL: (a) a therapeutically effective amount of the compound provided herein in combination with (b) a therapeutically effective amount of the bispecific antibody, wherein the bispecific antibody is glofitamab or mosunetuzumab.

### Non-Hodgkin Lymphoma

In certain embodiments, the NHL is B-cell non-Hodgkin lymphoma also referred to as B-cell lymphoma (BCL). BCL encompasses large B-cell lymphoma (LBCL) and follicular lymphoma (FL). In certain embodiments, the NHL is large B-cell lymphoma (LBCL). LBCL encompasses diffuse large B-cell lymphoma (DLBCL) and primary mediastinal B-cell lymphoma (PMBL).

In certain embodiments, the NHL is diffuse large B-cell lymphoma (DLBCL). In one embodiment, the DLBCL is primary DLBCL. In one embodiment, the DLBCL is activated B-cell-like DLBCL (ABC-DLBCL). In one embodiment, the DLBCL is germinal center B-cell-like DLBCL (GCB-DLBCL). In one embodiment, the DLBCL is unclassified DLBCL. In one embodiment, the DLBCL is primary mediastinal B-cell type DLBCL (PMBL DLBCL). In one embodiment, the DLBCL is double-hit DLBCL (DHIT DLBCL), also referred to as cMyc/Bcl-2 mutant DLBCL. In one embodiment, the DLBCL is triple-hit DLBCL (THIT DLBCL) also referred to as cMyc/Bcl2/Bcl6 rearrangement DLBCL. In one embodiment, the DLBCL is DLBCL not otherwise specified (NOS) and high-grade B-cell lymphoma with MYC and BLC2 and/or BCL6 rearrangements with DLBCL morphology.

In certain embodiments, the NHL is follicular lymphoma (FL).

In one embodiment, the NHL is characterized by the presence of an Enhancer of Zeste Homolog 2 (EZH2) mutation. In one embodiment, the FL is characterized by the presence of EZH2 mutation. In one embodiment, the EZH2 mutation is detected by an FDA-approved test. In one embodiment, the EZH2 mutation is identified using tumor samples by whole genome sequencing (WGS). In one embodiment, the EZH2 mutation is identified using formalin-fixed, paraffin-embedded tumor samples, by whole genome sequencing (WGS).

In certain embodiments, the NHL is relapsed or refractory NHL. In one embodiment, the NHL is relapsed NHL. In one embodiment, the NHL is refractory NHL.

In certain embodiments, the NHL subject has radiological evidence of progression after achieving a complete response (CR). In certain embodiments, the NHL subject has achieved less than a CR to most recent systemic therapy containing regimen, and has radiological evidence of active disease or disease progression or recurrence in less than or equal to 12 months of prior stem cell transplantation (SCT).

In certain embodiments, the NHL subject has failed one or more lines of therapy and is not a candidate for other therapy. In certain embodiments, the subject has received at least one prior therapy and is not eligible for any therapy other than the methods of treatment described herein. In certain embodiments, the subject has relapsed after or progressed on standard anticancer therapy.

In certain embodiments, the subject has received at least one prior line of therapy. In certain embodiments, the subject has received at least two prior lines of therapy. In certain embodiments, the subject has received one or more systemic regimens, wherein one or more of the systemic regimens comprise an anti-CD20 therapy (e.g., rituximab). In certain embodiments, the subject has received one to three systemic regimens, wherein at least one of the systemic regimens is an anti-CD20 therapy (e.g., rituximab).

In one embodiment, the NHL is relapsed or refractory DLBCL. In one embodiment, the DLBCL is relapsed DLBCL. In one embodiment, the DLBCL is refractory DLBCL. In one embodiment, the DLBCL is refractory to an anthracycline. In one embodiment, the DLBCL is resistant to an anthracycline. In one embodiment, the DLBCL is refractory to doxorubicin. In one embodiment, the DLBCL is resistant to doxorubicin.

In one embodiment, the DLBCL is treated with one or more prior lines of treatment. In one embodiment, the DLBCL is treated with two or more prior lines of treatment.

In one embodiment, the DLBCL is transformed lymphoma. In another embodiment, the DLBCL is not otherwise specified (NOS) DLBCL.

In one embodiment, a subject having relapsed or refractory DLBCL has received at least one prior line of therapy. In one embodiment, a subject having relapsed or refractory DLBCL has received at least two prior lines of therapy. In one embodiment, no more than one of the prior lines of therapy may be a treatment for lower grade lymphoma. In one embodiment, a subject having relapsed or refractory DLBCL has received at least one standard treatment regimen for DLBCL. In one embodiment, a subject having relapsed or refractory DLBCL has received one or more systemic regimens, wherein one or more of the systemic regimens comprise an anti-CD20 therapy (e.g., rituximab). In certain embodiments, the subject has received one to three systemic regimens, wherein at least one of the systemic regimens is an anti-CD20 therapy (e.g., rituximab).

In one embodiment, the NHL is relapsed or refractory FL. In one embodiment, the FL is relapsed FL. In one embodiment, the FL is refractory FL.

In one embodiment, the FL is treated with one or more prior lines of treatment. In one embodiment, the FL is treated with two or more prior lines of treatment.

In one embodiment, a subject having relapsed or refractory FL has received at least one prior line of therapy. In one embodiment, a subject having relapsed or refractory FL has received at least two prior lines of therapy. In one embodiment, a subject having relapsed or refractory FL has Grade 1, 2, 3a or 3b according to Groupe d'Etude des Lymphomes Folliculaires (GELF) criteria (National Comprehensive Cancer Network. NCCN Clinical Practice Guidelines in Oncology: B-cell Lymphomas; V.2.2018. 2018 Feb. 26; V.2), the entirety of which is incorporated herein by reference. In one embodiment, a subject having relapsed or refractory FL has received one or more systemic regimens, wherein one or more of the systemic regimens comprise an anti-CD20 therapy (e.g., rituximab). In certain embodiments, the subject has received one to three systemic regimens, wherein at least one of the systemic regimens is an anti-CD20 therapy (e.g., rituximab).

In certain embodiments, the NHL is newly diagnosed NHL. In certain embodiments, the NHL is newly diagnosed diffuse large B-cell lymphoma (DLBCL). In certain embodiments, the NHL is newly diagnosed follicular lymphoma (FL).

### Administration of the Combination Therapy

Compound A, A-S, or A-R, or an enantiomer or mixture of enantiomers, a tautomer, an isotopolog, a pharmaceutically acceptable salt, solvate, hydrate, co-crystal, clathrate, or polymorph thereof, in combination with the bispecific antibody, may be administered by oral, parenteral (e.g., intramuscular, intraperitoneal, intravenous, CIV, intracistemal injection or infusion, subcutaneous administration, or implant), inhalation, nasal, vaginal, rectal, sublingual, or topical (e.g., transdermal or local) routes of administration. Compound A, A-S, or A-R, or an enantiomer or mixture of enantiomers, a tautomer, an isotopolog, a pharmaceutically acceptable salt, solvate, hydrate, co-crystal, clathrate, or polymorph thereof, may be formulated alone or together with the bispecific antibody, in suitable dosage unit with pharmaceutically acceptable excipients, carriers, adjuvants and vehicles, appropriate for each route of administration. In one embodiment, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, in combination with the bispecific antibody, is administered orally.

In one embodiment, the bispecific antibody is glofitamab and the glofitamab, in combination with a compound provided herein, is administered intravenously, for example via intravenous infusion. In another embodiment, the bispecific antibody is mosunetuzumab and the mosunetuzumab, in combination with a compound provided herein, is administered subcutaneously or intravenously, for example via intravenous infusion. The bispecific antibody may be formulated alone or together with a compound provided herein, in suitable dosage unit with pharmaceutically acceptable excipients, carriers, adjuvants and vehicles, appropriate for each route of administration.

### Compound

Provided herein is Compound A, A-S, or A-R, or an enantiomer or mixture of enantiomers, a tautomer, an isotopolog, a pharmaceutically acceptable salt, solvate, hydrate, co-crystal, clathrate, or polymorph thereof for use in methods of treating and/or managing non-Hodgkin lymphoma in subjects.

In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof is administered in a therapeutically effective amount. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof is administered according to the locally approved label or pharmacy manual for preparation, administration, and storage information.

In certain embodiments, a therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is from about 0.01 mg to about 1.6 mg per day, from about 0.01 mg to about 1.2 mg per day, from about 0.01 mg to about 1.0 mg per day, from about 0.05 mg to about 0.9 mg per day, from about 0.05 mg to about 0.8 mg per day, from about 0.05 mg to about 0.7 mg per day, from about 0.1 mg to about 0.6 mg per day, from about 0.1 mg to about 0.5 mg per day, or from about 0.1 mg to about 0.4 mg per day. In some embodiments, a therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is from about 0.01 mg to about 1.6 mg per day. In some embodiments, a therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is from about 0.01 mg to about 1.2 mg per day. In some embodiments, a therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is from about 0.01 mg to about 1.0 mg per day. In some embodiments, a therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is from about 0.05 mg to about 0.9 mg per day. In some embodiments, a therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is from about 0.05 mg to about 0.8 mg per day. In some embodiments, a therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is from about 0.05 mg to about 0.7 mg per day. In some embodiments, a therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is from about 0.1 mg to about 0.6 mg per day. In some embodiments, a therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is from about 0.1 mg to about 0.5 mg per day. In some embodiments, a therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is from about 0.1 mg to about 0.4 mg per day.

In certain embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.11 mg, about 0.12 mg, about 0.13 mg, about 0.14 mg, about 0.15 mg, about 0.16 mg, about 0.17 mg, about 0.18 mg, about 0.19 mg, about 0.2 mg, about 0.25 mg, about 0.3 mg, about 0.35 mg, about 0.4 mg, about 0.45 mg, about 0.5 mg, about 0.55 mg, about 0.6 mg, about 0.65 mg, about 0.7 mg, about 0.75 mg, about 0.8 mg, about 0.85 mg, about 0.9 mg, about 0.95 mg, about 1 mg, about 1.05 mg, about 1.1 mg, about 1.15 mg, about 1.2 mg, about 1.25 mg, about 1.3 mg, about 1.35 mg, about 1.4 mg, about 1.45 mg, about 1.5 mg, about 1.55 mg, or about 1.6 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 0.01 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 0.05 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 0.1 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 0.15 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 0.2 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 0.25 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 0.3 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 0.35 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 0.4 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 0.45 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 0.5 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 0.55 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 0.6 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 0.65 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 0.7 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 0.75 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 0.8 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 0.85 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 0.9 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 0.95 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 1 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 1.1 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 1.2 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 1.3 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 1.4 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 1.5 mg per day. In some embodiments, the therapeutically or prophylactically effective amount of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is about 1.6 mg per day.

In one embodiment, the recommended daily dose range of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, for the conditions described herein lies within the range of from about 0.01 mg to about 1.6 mg per day, preferably given as a single once-a-day dose, or in divided doses throughout a day. In some embodiments, the dosage ranges from about 0.05 mg to about 0.9 mg per day. In other embodiments, the dosage ranges from about 0.1 mg to about 0.4 mg per day. Specific doses per day include 0.01 mg, 0.02 mg, 0.03 mg, 0.04 mg, 0.05 mg, 0.06 mg, 0.07 mg, 0.08 mg, 0.09 mg, 0.1 mg, 0.11 mg, 0.12 mg, 0.13 mg, 0.14 mg, 0.15 mg, 0.16 mg, 0.17 mg, 0.18 mg, 0.19 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1 mg, 1.05 mg, 1.1 mg, 1.15 mg, 1.2 mg, 1.25 mg, 1.3 mg, 1.35 mg, 1.4 mg, 1.45 mg, 1.5 mg, 1.55 mg, or 1.6 mg per day.

Compound A, A-S, or A-R, or an enantiomer or mixture of enantiomers, a tautomer, an isotopolog, a pharmaceutically acceptable salt, solvate, hydrate, co-crystal, clathrate, or polymorph thereof, in combination with the bispecific antibody provided herein, can be delivered as a single dose such as, e.g., a single bolus injection, or oral tablets or pills; or over time, such as, e.g., continuous infusion over time or divided bolus doses over time. Compound A, A-S, or A-R, or an enantiomer or mixture of enantiomers, a tautomer, an isotopolog, a pharmaceutically acceptable salt, solvate, hydrate, co-crystal, clathrate, or polymorph thereof, in combination with the bispecific antibody provided herein, can be administered repeatedly if necessary, for example, until the subject experiences stable disease or regression, or until the subject experiences disease progression or unacceptable toxicity.

Compound A, A-S, or A-R, or an enantiomer or mixture of enantiomers, a tautomer, an isotopolog, a pharmaceutically acceptable salt, solvate, hydrate, co-crystal, clathrate, or polymorph thereof, in combination with the bispecific antibody provided herein, can be administered once daily (QD), or divided into multiple daily doses such as twice daily (BID), three times daily (TID), and four times daily (QID). In addition, the administration can be continuous (i.e., daily for consecutive days) or intermittent, e.g., in cycles (i.e., including days, weeks, or months of rest without drug).

As used herein, the term “daily” is intended to mean that a therapeutic compound, such as Compound A, A-S, or A-R, is administered once or more than once each day, for example, for a period of time. The term “continuous” is intended to mean that a therapeutic compound, such as Compound A, A-S, or A-R, is administered daily for an uninterrupted period of at least 10 days to 52 weeks. The term “intermittent” or “intermittently” as used herein is intended to mean stopping and starting at either regular or irregular intervals. For example, intermittent administration of Compound A, A-S, or A-R is administration for one to six days per week, administration in cycles (e.g., daily administration for two to eight consecutive weeks, then a rest period with no administration for up to one week), or administration on alternate days. The term “cycle” or “cycling” as used herein is intended to mean that a therapeutic compound, such as Compound A, A-S, or A-R, is administered daily or continuously but with a rest period.

In some embodiments, the frequency of administration of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is in the range of about a daily dose to about a monthly dose. In certain embodiments, administration of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is once a day, twice a day, three times a day, four times a day, once every other day, twice a week, once every week, once every two weeks, once every three weeks, or once every four weeks.

In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered once per day from one day to six months, from one week to three months, from one week to four weeks, from one week to three weeks, or from one week to two weeks. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered once per day for 1 to 180 days, for 7 to 90 days, for 7 to 28 days, for 7 to 21 days, or for 10 to 14 days. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered once per day for one week, two weeks, three weeks, or four weeks. In one embodiment, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered once per day for one week. In another embodiment, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered once per day for two weeks. In yet another embodiment, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered once per day for three weeks. In still another embodiment, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered once per day for four weeks. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered once per day for 7 days, 10 days, 14 days, 21 days or 28 days. In one embodiment, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered once per day for 7 days. In another embodiment, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered once per day for 10 days. In yet another embodiment, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered once per day for 14 days. In still another embodiment, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered once per day for 21 days. In still another embodiment, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered once per day for 28 days.

In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered once per day for 10 days in a 21-day cycle. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered once per day for 10 days followed by 11 days of rest. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered once per day on days 1 to 10 in a 21-day cycle. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered once per day for 14 days in a 28-day cycle. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered once per day for 14 days followed by 14 days of rest. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered once per day on days 1 to 14 in a 28-day cycle.

In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered for one, two, three, four, five, six, seven, eight, nine, ten, eleven, twelve, thirteen, fourteen, fifteen or more cycles. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered for one cycle. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered for two cycles. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered for three cycles. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered for four cycles. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered for five cycles. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered for six cycles. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered for seven cycles. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered for eight cycles. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered for nine cycles. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered for ten cycles. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered for eleven cycles. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered for twelve cycles. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered for thirteen or more cycles.

In certain embodiments, the subject to be treated with one of the methods provided herein has not been treated with the bispecific antibody provided herein prior to the administration of Compound A, A-S, or A-R, or an enantiomer or mixture of enantiomers, a tautomer, an isotopolog, a pharmaceutically acceptable salt, solvate, hydrate, co-crystal, clathrate, or polymorph thereof. In certain embodiments, the subject to be treated with one of the methods provided herein has been treated with the bispecific antibody provided herein subsequent to the administration of Compound A, A-S, or A-R, or an enantiomer or mixture of enantiomers, a tautomer, an isotopolog, a pharmaceutically acceptable salt, solvate, hydrate, co-crystal, clathrate, or polymorph thereof. In certain embodiments, the subject to be treated with one of the methods provided herein has been treated with the bispecific antibody provided herein concurrently with the administration of Compound A, A-S, or A-R, or an enantiomer or mixture of enantiomers, a tautomer, an isotopolog, a pharmaceutically acceptable salt, solvate, hydrate, co-crystal, clathrate, or polymorph thereof.

In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered simultaneously, separately or sequentially to the bispecific antibody provided herein. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered concurrently with, prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after), the bispecific antibody provided herein. In certain embodiments, Compound A, A-S, or A-R, or an enantiomer or mixture of enantiomers, a tautomer, an isotopolog, a pharmaceutically acceptable salt, solvate, hydrate, co-crystal, clathrate, or polymorph thereof, in combination with the bispecific antibody, can be administered on an alternating dosing schedule, with or without a resting period (e.g., no therapeutic agent is administered on certain days of the schedule). In certain embodiments, the administration of Compound A, A-S, or A-R, or an enantiomer or mixture of enantiomers, a tautomer, an isotopolog, a pharmaceutically acceptable salt, solvate, hydrate, co-crystal, clathrate, or polymorph thereof, in combination with the bispecific antibody includes, but is not limited to, sequential administration and concomitant administration.

### Bispecific Antibody

Provided herein is a bispecific antibody for use in methods of treating and/or managing non-Hodgkin lymphoma in subjects.

In certain embodiments, the bispecific antibody is administered in a therapeutically effective amount. In certain embodiments, the bispecific antibody is administered according to the locally approved label or pharmacy manual for preparation, administration, and storage information. In one embodiment, the bispecific antibody is glofitamab administered according to the label of Columvi®. In another embodiment, the bispecific antibody is mosunetuzumab administered according to the label of Lunsumio®.

The bispecific antibody, in combination with a compound provided herein, can be delivered as a single dose such as, e.g., a single bolus injection; or over time, such as, e.g., continuous infusion over time or divided bolus doses over time. The bispecific antibody, in combination with a compound provided herein, can be administered repeatedly if necessary, for example, until the subject experiences stable disease or regression, or until the subject experiences disease progression or unacceptable toxicity. The bispecific antibody, in combination with a compound provided herein, can be administered once daily (QD), or divided into multiple daily doses such as twice daily (BID), three times daily (TID), and four times daily (QID).

In certain embodiments, the bispecific antibody is glofitamab. In one embodiment, glofitamab is administered intravenously. In one embodiment, glofitamab is administered via intravenous infusion. In certain embodiments, the bispecific antibody is mosunetuzumab. In one embodiment, mosunetuzumab is administered subcutaneously.

### Obinutuzumab

Provided herein is obinutuzumab for use in methods of treating and/or managing non-Hodgkin lymphoma in subjects.

In certain embodiments, obinutuzumab is administered in a therapeutically effective amount. In certain embodiments, obinutuzumab is administered according to the locally approved label or pharmacy manual for preparation, administration, and storage information. In one embodiment, obinutuzumab is administered according to the label of Gazyva® or Gazyvaro®.

In one embodiment, obinutuzumab is administered intravenously. In one embodiment, obinutuzumab is administered via intravenous infusion.

### Dosage Regimens

In certain embodiments, the bispecific antibody is glofitamab administered in a 21-day priming dose cycle prior to administration of the compound. In certain embodiments, obinutuzumab is administered on day 1 and glofitamab is administered on days 8 and 15 in a 21-day priming dose cycle prior to administration of the compound. In certain embodiments, obinutuzumab is administered intravenously on day 1 in an amount of about 1,000 mg, and glofitamab is administered intravenously on day 8 in an amount of about 2.5 mg and on day 15 in an amount of about 10 mg in a 21-day priming dose cycle prior to administration of the compound. In certain embodiments, the bispecific antibody is glofitamab administered in two 21-day priming dose cycles prior to administration of the compound. In certain embodiments, obinutuzumab is administered on day 1 and glofitamab is administered on days 8 and 15 in a first 21-day priming dose cycle, and glofitamab is administered on day 1 in a second 21-day priming dose cycle, prior to administration of the compound. In certain embodiments, obinutuzumab is administered intravenously on day 1 in an amount of about 1,000 mg, and glofitamab is administered intravenously on day 8 in an amount of about 2.5 mg and on day 15 in an amount of about 10 mg in a first 21-day priming dose cycle, and glofitamab is administered intravenously on day 1 in an amount of about 30 mg in a second 21-day priming dose cycle, prior to administration of the compound. In certain embodiments, following any priming dose cycle(s), if any, glofitamab is administered once in a 21-day treatment cycle, while Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered for 10 days in the 21-day treatment cycle, namely administered for 10 days followed by 11 days of rest. In certain embodiments, following any priming dose cycle(s), if any, glofitamab is administered on day 1 in a 21-day treatment cycle, while Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered on days 1 to 10 in the 21-day treatment cycle. In certain embodiments, following any priming dose cycle(s), if any, glofitamab is administered intravenously on day 1 in an amount of about 30 mg in a 21-day treatment cycle, while Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered orally on days 1 to 10 in an amount of about 0.1 mg, about 0.2 mg or about 0.4 mg per day in the 21-day treatment cycle. In certain embodiments, glofitamab and Compound A, A-S, or A-R, or an enantiomer or mixture of enantiomers, a tautomer, an isotopolog, a pharmaceutically acceptable salt, solvate, hydrate, co-crystal, clathrate, or polymorph thereof, are administered in 12 cycles. In certain embodiments, glofitamab and Compound A, A-S, or A-R, or an enantiomer or mixture of enantiomers, a tautomer, an isotopolog, a pharmaceutically acceptable salt, solvate, hydrate, co-crystal, clathrate, or polymorph thereof, are administered in two 21-day priming dose cycles and ten 21-day treatment cycles, or one 21-day priming dose cycle and eleven 21-day treatment cycles, or no priming dose cycles and twelve 21-day treatment cycles.

In certain embodiments, the bispecific antibody is mosunetuzumab administered in a 21-day priming dose cycle prior to administration of the compound. In certain embodiments, mosunetuzumab is administered on days 1, 8 and 15 in a 21-day priming dose cycle prior to administration of the compound. In certain embodiments, mosunetuzumab is administered subcutaneously on day 1 in an amount of about 5 mg, on day 8 in an amount of about 45 mg, and on day 15 in an amount of about 45 mg in a 21-day priming dose cycle prior to administration of the compound. In certain embodiments, following any priming dose cycle(s), if any, mosunetuzumab is administered once in a 28-day treatment cycle, while Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered for 14 days in the 28-day treatment cycle, namely administered for 14 days followed by 14 days of rest. In certain embodiments, following any priming dose cycle(s), if any, mosunetuzumab is administered on day 1 in a 28-day treatment cycle, while Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered on days 1 to 14 in the 28-day treatment cycle. In certain embodiments, following any priming dose cycle(s), if any, mosunetuzumab is administered subcutaneously on day 1 in an amount of about 45 mg in a 28-day treatment cycle, while Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered orally on days 1 to 14 in an amount of about 0.1 mg, about 0.2 mg or about 0.4 mg per day in the 28-day treatment cycle. In certain embodiments, mosunetuzumab and Compound A, A-S, or A-R, or an enantiomer or mixture of enantiomers, a tautomer, an isotopolog, a pharmaceutically acceptable salt, solvate, hydrate, co-crystal, clathrate, or polymorph thereof, are administered in 12 cycles. In certain embodiments, mosunetuzumab and Compound A, A-S, or A-R, or an enantiomer or mixture of enantiomers, a tautomer, an isotopolog, a pharmaceutically acceptable salt, solvate, hydrate, co-crystal, clathrate, or polymorph thereof, are administered in one 21-day priming dose cycle and eleven 28-day treatment cycles, or no priming dose cycles and twelve 28-day treatment cycles.

In certain embodiments, premedication is administered before bispecific antibody dosing to prevent unwanted adverse events. In certain embodiments, the premedication is administered about 30 minutes, about 45 minutes, about 60 minutes, about 75 minutes, or about 90 minutes prior to the first, second and/or third doses of the bispecific antibody. In certain embodiments, the premedication includes granulocyte colony-stimulating factor (G-CSF) (e.g., 4-12 mcg/kg filgrastim SC or IV, or 4-8 mg pegfilgrastim SC or IV), dexamethasone (e.g., about 20 mg IV or PO), methylprednisolone (e.g., about 80 mg IV or PO), prednisone (e.g., about 100 mg IV), prednisolone (e.g., about 100 mg IV), diphenhydramine (e.g., 50-100 mg PO) and/or paracetamol (e.g., 500-1,000 mg PO).

### Specific Embodiments

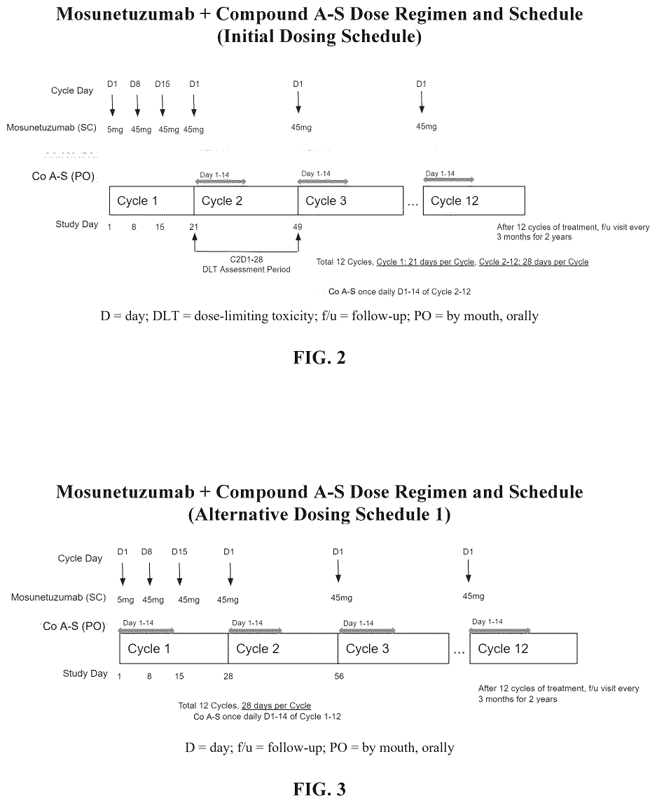

In certain embodiments, the combination therapy is administered as illustrated in FIG. 2-6. In certain embodiments, the combination therapy is administered in multiple 21-day or 28-day cycles or a combination thereof. In certain embodiments, the combination therapy is administered in twelve cycles.

In certain embodiments, the combination therapy is administered as illustrated in FIG. 2. In certain embodiments, mosunetuzumab is administered on days 1, 8 and 15 in a 21-day priming dose cycle (C1) prior to administration of the compound. In certain embodiments, in the second to twelfth cycles (C2-C12), mosunetuzumab is administered on day 1 in a 28-day cycle, while Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered on days 1 to 14 in the 28-day cycle.

In certain embodiments, mosunetuzumab is administered subcutaneously on day 1 in an amount of about 5 mg, on day 8 in an amount of about 45 mg, and on day 15 in an amount of about 45 mg in a 21-day priming dose cycle (C1) prior to administration of the compound. In certain embodiments, in the second to twelfth cycles (C2-C12), mosunetuzumab is administered subcutaneously on day 1 in an amount of about 45 mg in a 28-day cycle, while Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered orally in an amount of about 0.1 mg, about 0.2 mg or about 0.4 mg per day on days 1 to 14 in the 28-day cycle.

In certain embodiments, the combination therapy is administered as illustrated in FIG. 3. In certain embodiments, in a first cycle (C1), mosunetuzumab is administered on days 1, 8 and 15 in a 28-day cycle, while Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered on days 1 to 14 in the 28-day cycle. In certain embodiments, in the second to twelfth cycles (C2-C12), mosunetuzumab is administered on day 1 in a 28-day cycle, while Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered on days 1 to 14 in the 28-day cycle.

In certain embodiments, in a first cycle (C1), mosunetuzumab is administered subcutaneously on day 1 in an amount of about 5 mg, on day 8 in an amount of about 45 mg, and on day 15 in an amount of about 45 mg in a 28-day cycle, while Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered orally in an amount of about 0.1 mg, about 0.2 mg or about 0.4 mg per day on days 1 to 14 in the 28-day cycle. In certain embodiments, in the second to twelfth cycles (C2-C12), mosunetuzumab is administered subcutaneously on day 1 in an amount of about 45 mg in a 28-day cycle, while Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered orally in an amount of about 0.1 mg, about 0.2 mg or about 0.4 mg per day on days 1 to 14 in the 28-day cycle.

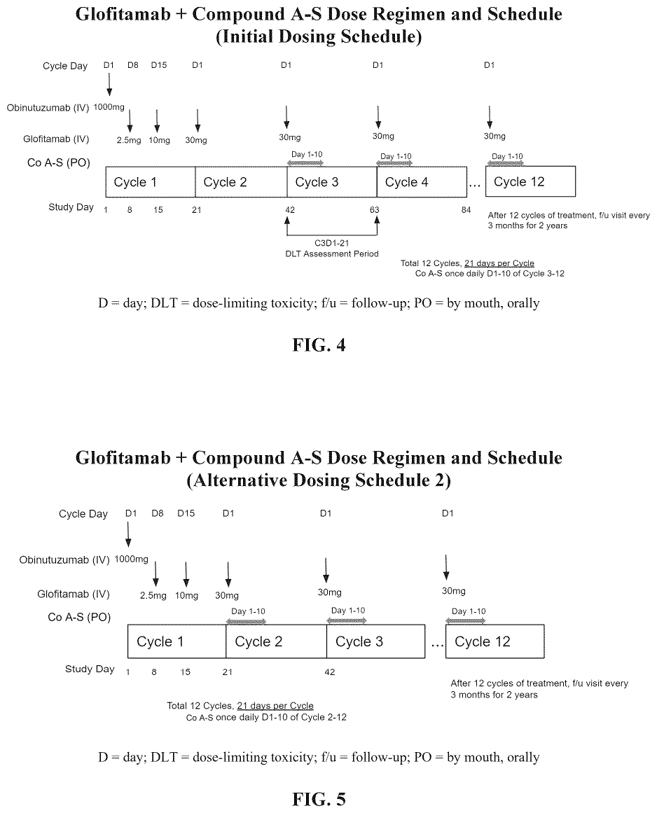

In certain embodiments, the combination therapy is administered as illustrated in FIG. 4. In certain embodiments, obinutuzumab is administered on day 1 and glofitamab is administered on days 8 and 15 in a first 21-day priming dose cycle (C1) prior to administration of the compound. In certain embodiments, glofitamab is administered on day 1 in a second 21-day priming dose cycle (C2) prior to administration of the compound. In certain embodiments, in the third to twelfth cycles (C3-C12), glofitamab is administered on day 1 in a 21-day cycle, while Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered on days 1 to 10 in the 21-day cycle.

In certain embodiments, obinutuzumab is administered intravenously on day 1 in an amount of about 1,000 mg, and glofitamab is administered intravenously on day 8 in an amount of about 2.5 mg and on day 15 in an amount of about 10 mg in a first 21-day priming dose cycle (C1) prior to administration of the compound. In certain embodiments, glofitamab is administered intravenously on day 1 in an amount of about 30 mg in a second 21-day priming dose cycle (C2) prior to administration of the compound. In certain embodiments, in the third to twelfth cycles (C3-C12), glofitamab is administered intravenously on day 1 in an amount of about 30 mg in a 21-day cycle, while Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered orally in an amount of about 0.1 mg, about 0.2 mg or about 0.4 mg per day on days 1 to 10 in the 21-day cycle.

In certain embodiments, the combination therapy is administered as illustrated in FIG. 5. In certain embodiments, obinutuzumab is administered on day 1 and glofitamab is administered on days 8 and 15 in a 21-day priming dose cycle (C1) prior to administration of the compound. In certain embodiments, in the second to twelfth cycles (C2-C12), glofitamab is administered on day 1 in a 21-day cycle, while Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered on days 1 to 10 in the 21-day cycle.

In certain embodiments, obinutuzumab is administered intravenously on day 1 in an amount of about 1,000 mg, and glofitamab is administered intravenously on day 8 in an amount of about 2.5 mg and on day 15 in an amount of about 10 mg in a 21-day priming dose cycle (C1) prior to administration of the compound. In certain embodiments, in the second to twelfth cycles (C2-C12), glofitamab is administered intravenously on day 1 in an amount of about 30 mg in a 21-day cycle, while Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered orally in an amount of about 0.1 mg, about 0.2 mg or about 0.4 mg per day on days 1 to 10 in the 21-day cycle.

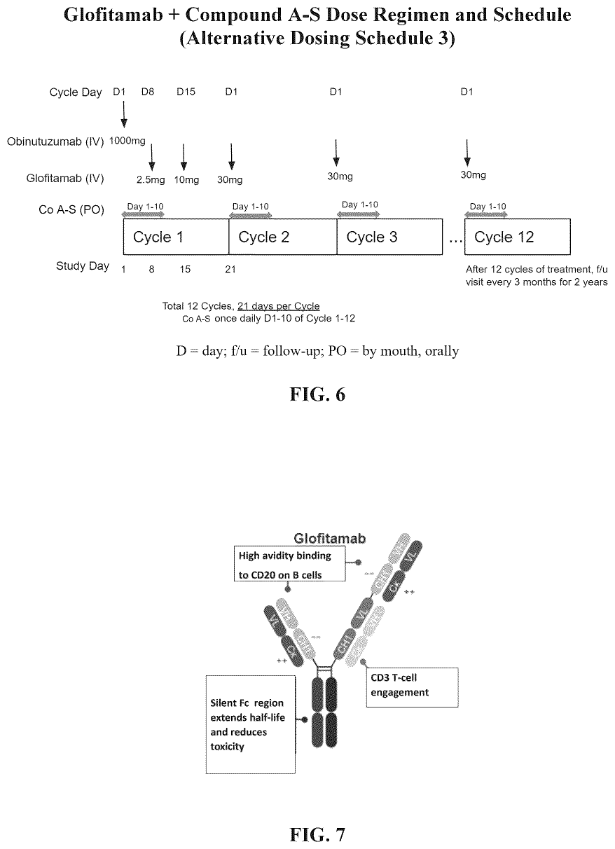

In certain embodiments, the combination therapy is administered as illustrated in FIG. 6. In certain embodiments, in a first cycle (C1), obinutuzumab is administered on day 1 and glofitamab is administered on days 8 and 15 in a 21-day cycle, while Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered on days 1 to 10 in the 21-day cycle. In certain embodiments, in the second to twelfth cycles (C2-C12), glofitamab is administered on day 1 in a 21-day cycle, while Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered on days 1 to 10 in the 21-day cycle.

In certain embodiments, in a first cycle (C1), obinutuzumab is administered intravenously on day 1 in an amount of about 1,000 mg, and glofitamab is administered intravenously on day 8 in an amount of about 2.5 mg and on day 15 in an amount of about 10 mg in a 21-day cycle, while Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered orally in an amount of about 0.1 mg, about 0.2 mg or about 0.4 mg per day on days 1 to 10 in the 21-day cycle. In certain embodiments, in the second to twelfth cycles (C2-C12), glofitamab is administered intravenously on day 1 in an amount of about 30 mg in a 21-day cycle, while Compound A, A-S, or A-R, or an enantiomer or mixture of enantiomers, a tautomer, an isotopolog, a pharmaceutically acceptable salt, solvate, hydrate, co-crystal, clathrate, or polymorph thereof, is administered orally in an amount of about 0.1 mg, about 0.2 mg or about 0.4 mg per day on days 1 to 10 in the 21-day cycle.

### Pharmaceutical Compositions and Dosage Forms

In one embodiment, provided herein are pharmaceutical compositions and dosage forms, which comprise Compound A, A-S, or A-R, or an enantiomer or mixture of enantiomers, a tautomer, an isotopolog, a pharmaceutically acceptable salt, solvate, hydrate, co-crystal, clathrate, or polymorph thereof, and which can be administered in combination with the bispecific antibody provided herein. In another embodiment, the pharmaceutical compositions and dosage forms further comprise one or more excipients.

In one embodiment, provided herein are pharmaceutical compositions and dosage forms, which comprise the bispecific antibody provided herein, and which can be administered in combination with Compound A, A-S, or A-R, or an enantiomer or mixture of enantiomers, a tautomer, an isotopolog, a pharmaceutically acceptable salt, solvate, hydrate, co-crystal, clathrate, or polymorph thereof. In another embodiment, the pharmaceutical compositions and dosage forms further comprise one or more excipients.

In one embodiment, provided herein are pharmaceutical compositions and dosage forms, which comprise (i) Compound A, A-S, or A-R, or an enantiomer or mixture of enantiomers, a tautomer, an isotopolog, a pharmaceutically acceptable salt, solvate, hydrate, co-crystal, clathrate, or polymorph thereof, and (ii) the bispecific antibody provided herein. In another embodiment, the pharmaceutical compositions and dosage forms further comprise one or more excipients.

In certain embodiments, pharmaceutical compositions and dosage forms provided herein also comprise one or more additional active agents in amounts effective for achieving a modulation of disease or disease symptoms, including those described herein.

In certain embodiments, the pharmaceutical compositions provided herein may be administered orally, parenterally, by inhalation spray, topically, rectally, nasally, buccally, vaginally or via an implanted reservoir, preferably by oral or parenteral administration. Oral delivery formats include, but are not limited to, tablets, capsules, caplets, solutions, suspensions, and syrups, and may also comprise a plurality of granules, beads, powders or pellets that may or may not be encapsulated. In one embodiment, the pharmaceutical compositions may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

In certain embodiments, dosage forms provided herein for Compound A, A-S, or A-R, or an enantiomer or mixture of enantiomers, a tautomer, an isotopolog, a pharmaceutically acceptable salt, solvate, hydrate, co-crystal, clathrate, or polymorph thereof, are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal, or transcutaneous administration to a subject. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a subject, including suspensions (e.g., aqueous or non-aqueous liquid suspensions, oil-in-water emulsions, or water-in-oil liquid emulsions), solutions, and elixirs; liquid dosage forms suitable for parenteral administration to a subject; eye drops or other ophthalmic preparations suitable for topical administration; and sterile solids (e.g., crystalline or amorphous solids) that can be reconstituted to provide liquid dosage forms suitable for parenteral administration to a subject. Certain pharmaceutical compositions and formulations of Compound A-S are described in U.S. Pat. No. 11,504,378, the entirety of which is incorporated herein by reference.

In certain embodiments, dosage forms provided herein for the bispecific antibody, are suitable for oral, mucosal (e.g., nasal, sublingual, vaginal, buccal, or rectal), parenteral (e.g., subcutaneous, intravenous, bolus injection, intramuscular, or intraarterial), topical (e.g., eye drops or other ophthalmic preparations), transdermal, or transcutaneous administration to a subject. Examples of dosage forms include, but are not limited to: tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a subject, including suspensions (e.g., aqueous or non-aqueous liquid suspensions, oil-in-water liquid emulsions, or water-in-oil liquid emulsions), solutions, and elixirs; liquid dosage forms suitable for parenteral administration to a subject; eye drops or other ophthalmic preparations suitable for topical administration; and sterile solids (e.g., crystalline or amorphous solids) that can be reconstituted to provide liquid dosage forms suitable for parenteral administration to a subject. In one embodiment, the bispecific antibody is glofitamab formulated as described in the package insert of Columvi®. In one embodiment, the bispecific antibody is glofitamab formulated for administration to subjects by intravenous administration such as intravenous infusion. In one embodiment, the bispecific antibody is mosunetuzumab formulated as described in the package insert of Lunsumio®. In one embodiment, the bispecific antibody is mosunetuzumab formulated for administration to subjects by subcutaneous injection.

Whether a particular excipient is suitable for incorporation into a pharmaceutical composition or dosage form provided herein depends on a variety of factors, including, but not limited to, the route of administration. For example, oral dosage forms such as tablets may contain excipients not suited for use in parenteral dosage forms. The suitability of a particular excipient may also depend on the specific active ingredient(s) in the dosage form. For example, the decomposition of some active ingredients may be accelerated by some excipients such as lactose, or when exposed to water. Active ingredients that comprise primary or secondary amines are particularly susceptible to such accelerated decomposition. Consequently, encompassed herein are pharmaceutical compositions and dosage forms that contain little, if any, lactose. As used herein, the term “lactose-free” means that the amount of lactose present, if any, is insufficient to substantially increase the degradation rate of an active ingredient.

Lactose-free compositions provided herein can comprise excipients that are listed, for example, in the U.S. Pharmacopeia (USP) 25-NF20 (2002). In certain embodiments, lactose-free compositions comprise active ingredient(s), a binder/filler, and a lubricant in pharmaceutically compatible and pharmaceutically acceptable amounts. In certain embodiments, lactose-free dosage forms comprise active ingredient(s), microcrystalline cellulose, pre-gelatinized starch, and magnesium stearate.

Further encompassed herein are anhydrous pharmaceutical compositions and dosage forms comprising active ingredient(s), since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2nd ed., Marcel Dekker, NY, NY, 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, in certain embodiments, provided herein are anhydrous compositions packaged using materials to prevent exposure to water such that they can be included in suitable formulary kits. Examples of suitable packaging include, but are not limited to, hermetically sealed foils, plastics, unit dose containers (e.g., vials), blister packs, and strip packs.

Encompassed herein are pharmaceutical compositions and dosage forms that comprise one or more compounds that reduce the rate by which an active ingredient will decompose. Such compounds, which are referred to herein as “stabilizers”, include, but are not limited to, antioxidants such as ascorbic acid, pH buffers, or salt buffers.

### Oral Dosage Forms

In certain embodiments, pharmaceutical compositions provided herein that are suitable for oral administration are formulated as discrete dosage forms, examples of which include, but are not limited to, tablets (e.g., chewable tablets), caplets, capsules, and liquids (e.g., flavored syrups). Such dosage forms contain predetermined amounts of active ingredient(s) and may be prepared by known methods of pharmacy. See generally, Remington's Pharmaceutical Sciences, 18th ed., Mack Publishing, Easton PA (1990).

In certain embodiments, the oral dosage forms provided herein are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, microcrystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms may be prepared by known methods of pharmacy. In certain embodiments, pharmaceutical compositions and dosage forms are prepared by uniformly and intimately admixing the active ingredient(s) with liquid carriers, finely divided solid carriers, or both, and then shaping the product into the desired presentation if necessary.

In certain embodiments, a tablet is prepared by compression or molding. In certain embodiments, compressed tablets are prepared by compressing in a suitable machine the active ingredient(s) in a free-flowing form, e.g., powder or granules, optionally mixed with an excipient. In certain embodiments, molded tablets are made by molding in a suitable machine a mixture of a powdered compound moistened with an inert liquid diluent.

Examples of excipients that can be used in oral dosage forms provided herein include, but are not limited to, binders, fillers, disintegrants, and lubricants. Binders suitable for use in pharmaceutical compositions and dosage forms provided herein include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., methyl cellulose, ethyl cellulose, cellulose acetate, carboxymethyl cellulose calcium, sodium carboxymethyl cellulose), polyvinyl pyrrolidone, pre-gelatinized starch, hydroxypropyl methyl cellulose (e.g., Nos. 2208, 2906, 2910), microcrystalline cellulose, and mixtures thereof. Suitable forms of microcrystalline cellulose include, but are not limited to, AVICEL-PH-101™, AVICEL-PH-103™, AVICEL RC-581™, AVICEL-PH-105™ (FMC Corporation, American Viscose Division, Avicel Sales, Marcus Hook, PA), and mixtures thereof. A specific binder is a mixture of microcrystalline cellulose and sodium carboxymethyl cellulose (e.g., AVICEL RC-581™). Suitable anhydrous or low moisture excipients or additives include AVICEL-PH-103™ and Starch 1500 LM™.

Examples of fillers suitable for use in the pharmaceutical compositions and dosage forms provided herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. In certain embodiments, the binder or filler in pharmaceutical compositions provided herein is present in from about 50 to about 99 weight percent of the pharmaceutical composition or dosage form.

Disintegrants are used in the compositions provided herein to provide tablets the ability to disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredient(s) should be used to form solid oral dosage forms provided herein. The amount of disintegrant used varies based upon the type of formulation. In certain embodiments, the pharmaceutical compositions provided herein comprise from about 0.5 to about 15 weight percent or from about 1 to about 5 weight percent of disintegrant. Disintegrants that are suitable for use in pharmaceutical compositions and dosage forms provided herein include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that are suitable for use in pharmaceutical compositions and dosage forms provided herein include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, but are not limited to, a syloid silica gel (AEROSIL200™, W. R. Grace Co., Baltimore, MD), a coagulated aerosol of synthetic silica (Degussa Co. of Plano, TX), CAB-O-SIL™ (a pyrogenic silicon dioxide, Cabot Co. of Boston, MA), and mixtures thereof. In certain embodiments, if used at all, lubricants are used in an amount of less than about 1 weight percent of the pharmaceutical compositions or dosage forms into which they are incorporated.

In certain embodiments, provided herein is a solid oral dosage form, comprising Compound A, A-S, or A-R, or an enantiomer or mixture of enantiomers, a tautomer, an isotopolog, a pharmaceutically acceptable salt, solvate, hydrate, co-crystal, clathrate, or polymorph thereof, and one or more excipients selected from anhydrous lactose, microcrystalline cellulose, polyvinylpyrrolidone, stearic acid, colloidal anhydrous silica, and gelatin.

In certain embodiments, provided herein is a solid oral dosage form, comprising Compound A, A-S, or A-R, or an enantiomer or mixture of enantiomers, a tautomer, an isotopolog, a pharmaceutically acceptable salt, solvate, hydrate, co-crystal, clathrate, or polymorph thereof, and anhydrous lactose, microcrystalline cellulose, polyvinylpyrrolidone, stearic acid, colloidal anhydrous silica, and gelatin.

In certain embodiments, provided herein is a solid oral dosage form, comprising a hydrochloride salt of Compound A, A-S, or A-R, or an enantiomer or mixture of enantiomers, a tautomer, an isotopolog, a pharmaceutically acceptable solvate, hydrate, co-crystal, clathrate, or polymorph thereof, and one or more excipients selected from anhydrous lactose, microcrystalline cellulose, polyvinylpyrrolidone, stearic acid, colloidal anhydrous silica, and gelatin.

In certain embodiments, provided herein is a solid oral dosage form, comprising a hydrochloride salt of Compound A, A-S, or A-R, or an enantiomer or mixture of enantiomers, a tautomer, an isotopolog, a pharmaceutically acceptable solvate, hydrate, co-crystal, clathrate, or polymorph thereof, and anhydrous lactose, microcrystalline cellulose, polyvinylpyrrolidone, stearic acid, colloidal anhydrous silica, and gelatin.

### Delayed Release Dosage Forms

In certain embodiments, the active ingredient(s) provided herein are administered by controlled release means or by delivery devices. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,566; each of which is incorporated herein by reference in its entirety. In certain embodiments, such dosage forms are used to provide slow or controlled-release of one or more active ingredients using, for example, hydroxypropyl methyl cellulose, other polymer matrices, gels, permeable membranes, osmotic systems, multilayer coatings, microparticles, liposomes, microspheres, or a combination thereof to provide the desired release profile in varying proportions. Encompassed herein are single unit dosage forms suitable for oral administration, including, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions.

### Parenteral Dosage Forms

Parenteral dosage forms can be administered to subjects by various routes including, but not limited to, subcutaneous, intravenous (including intravenous infusion and bolus injection), intramuscular, and intraarterial. Because their administration typically bypasses subjects' natural defenses against contaminants, parenteral dosage forms are preferably sterile or capable of being sterilized prior to administration to a subject. Examples of parenteral dosage forms include, but are not limited to, solutions ready for infusion or injection, dry products ready to be dissolved or suspended in a pharmaceutically acceptable vehicle for infusion or injection, suspensions ready for infusion or injection, and emulsions. In certain embodiments, the bispecific antibody is glofitamab in solution for administration to subjects by intravenous infusion. In certain embodiments, the bispecific antibody is mosunetuzumab in solution for administration to subjects by subcutaneous injection.

Some suitable vehicles that can be used to provide parenteral dosage forms provided herein include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate. Compounds that increase the solubility of one or more of the active ingredients disclosed herein can also be incorporated into the parenteral dosage forms provided herein.

In certain embodiments, provided herein is a parenteral dosage form suitable for subcutaneous administration, comprising mosunetuzumab and one or more excipients selected from L-histidine, L-methionine, acetic acid, sucrose, polysorbate 20 (E432), and water for injection.

In certain embodiments, provided herein is a parenteral dosage form suitable for subcutaneous administration, comprising mosunetuzumab, L-histidine, L-methionine, acetic acid, sucrose, polysorbate 20 (E432), and water for injection.

In certain embodiments, provided herein is a parenteral dosage form suitable for intravenous administration, comprising glofitamab and one or more excipients selected from L-histidine, L-histidine hydrochloride monohydrate, L-methionine, sucrose, polysorbate 20 (E432), and water for injection.

In certain embodiments, provided herein is a parenteral dosage form suitable for intravenous administration, comprising glofitamab, L-histidine, L-histidine hydrochloride monohydrate, L-methionine, sucrose, polysorbate 20 (E432), and water for injection.

### Kits

In certain embodiments, active ingredients provided herein are not administered to a subject at the same time or by the same route of administration. Therefore, encompassed herein are kits which, when used by the medical practitioner, can simplify the administration of appropriate amounts of active ingredients to a subject. In certain embodiments, a kit provided herein comprises a dosage form of Compound A, A-S, or A-R, or an enantiomer or mixture of enantiomers, a tautomer, an isotopolog, a pharmaceutically acceptable salt, solvate, hydrate, co-crystal, clathrate, or polymorph thereof. In certain embodiments, the kit provided herein further comprises the bispecific antibody provided herein. In certain embodiments, the kit provided herein further comprises a device that is used to administer the active ingredient(s). Examples of such devices include, but are not limited to, syringes, drip bags, patches, and inhalers.

In certain embodiments, the kit provided herein further comprises pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

### EXAMPLES

Certain embodiments of the invention are illustrated by the following non-limiting examples.

### Example 1: A Phase 1b, Open-Label Study of Compound A-S in Combination with Glofitamab or Mosunetuzumab in Participants with B-Cell Non-Hodgkin Lymphoma Study Rationale

The Example is to evaluate the safety, efficacy, pharmacokinetics (PK), and pharmacodynamics (PD) of Compound A-S in combination with glofitamab or mosunetuzumab in participants with B-cell non-Hodgkin lymphoma (BCL). This study includes an initial dose escalation phase (PART 1), comprised of several dose escalation cohorts, designed to evaluate multiple dose levels of Compound A-S in its respective treatment combinations with fixed treatment regimens of glofitamab or mosunetuzumab. The initial dose escalation phase will be followed by an expansion phase (PART 2) incorporating a randomized dose optimization strategy to identify the RP2D for Compound A-S.

### Objectives and Endpoints

### Objectives and Endpoints for Dose Escalation

Primary Objectives

| Safety Objectives | Corresponding Endpoint |
|---|---|
| To evaluate the safety of Compound A-S in | DLTs |
| combination with mosunetuzumab | Incidence, nature, and severity of physical findings and |
| To evaluate the safety of Compound A-S in | adverse events, with severity determined according to |
| combination with glofitamab | the NCI CTCAE v5.0; for CRS, ICANS and HLH, |
| | severity determined according to the ASTCT grading |
| | criteria (Lee et al. 2019; Hines et al. 2023) |
| | Change from baseline in targeted vital signs |
| | Change from baseline in targeted clinical laboratory |
| | test results |
| | Tolerability, as assessed by the incidence of dose |
| | interruptions, dose reductions, and dose intensity, and |
| | treatment discontinuation not due to efficacy events |

Secondary Objectives

| Efficacy Objectives | Corresponding Endpoints |
|---|---|
| To evaluate the preliminary efficacy of | Best CR rate, defined as the proportion of participants |
| mosunetuzumab in combination with Compound A-S | whose best overall response is a CR during the study, |
| To evaluate the preliminary efficacy of glofitamab | as determined by the investigator using Lugano 2014 |
| in combination with Compound A-S | Criteria |
| | Best ORR (CR or PR at any time) on study as |
| | determined by the investigator using Lugano 2014 |
| | Criteria |
| | DOR, defined as the time from the first occurrence of a |
| | documented objective response (CR or PR) to disease |
| | progression or relapse, as determined by the |
| | investigator using Lugano 2014 Criteria, or death from |
| | any cause, whichever occurs first |

ASTCT = American Society for Transplantation and Cellular Therapy; CR = complete response; CRS = cytokine-release syndrome; CTCAE = Common Terminology Criteria for Adverse Events; DLT = dose-limiting toxicity; DOR = duration of response; ICANS = immune effector cell-associated neurotoxicity syndrome; NCI = National Cancer Institute; ORR = overall response rate; PR = partial response.

### Objectives and Endpoints for Dose Expansion

Primary Objectives

| Efficacy and Safety Objectives | Corresponding Endpoint |
|---|---|
| To select the RP2D of Compound A-S in | Best ORR, defined as the proportion of participants whose best |
| combination with mosunetuzumab | overall response is a PR or a CR during the study, as |
| To select the RP2D of Compound A-S in | determined by the investigator using Lugano 2014 Criteria |
| combination with glofitamab | Incidence, nature, and severity of physical findings and adverse |
| | events, with severity determined according to the NCI CTCAE |
| | v5.0; for CRS, ICANS and HLH, severity determined according |
| | to the ASTCT grading criteria (Lee et al. 2019; Hines et al. |
| | 2023) |
| | Tolerability, as assessed by the incidence of dose interruptions, |
| | dose reductions, and dose intensity, and treatment |
| | discontinuation not due to efficacy events |

Secondary Objectives

| Efficacy Objectives | Corresponding Endpoints |
|---|---|
| To evaluate the efficacy of | Best CR rate, defined as the proportion of participants whose |
| mosunetuzumab in combination with | best overall response is a CR during the study, as determined by |
| Compound A-S | the investigator using Lugano 2014 Criteria |
| To evaluate the efficacy of glofitamab in | DOR, defined as the time from the first occurrence of a |
| combination with Compound A-S | documented objective response (CR or PR) to disease |
| | progression or relapse, as determined by the investigator using |
| | Lugano 2014 Criteria, or death from any cause, whichever |
| | occurs first |
| | PFS, defined as the time from the first study treatment to the |
| | first occurrence of disease progression or relapse, as determined |
| | by the investigator using Lugano 2014 Criteria, or death from |
| | any cause, whichever occurs first |
| | EFS, defined as the time from the first study treatment to the |
| | first occurrence of disease progression or relapse, as determined |
| | by the investigator using Lugano 2014 Criteria, initiation of |
| | NALT, or death from any cause, whichever occurs first |
| | OS, defined as the time from the first study treatment to death |
| | from any cause |
| To evaluate the safety of | Occurrence and severity of adverse events, with severity |
| mosunetuzumab SC in combination with | determined according to NCI CTCAE v5.0 grading scale. For |
| Compound A-S in Cohort E | CRS, severity determined according to the ASTCT CRS |
| To evaluate the safety of glofitamab IV in | Consensus Grading criteria (Lee et al. 2019). |
| combination with Compound A-S in | |
| Cohort F | |

ADA = anti-drug antibody; ASTCT = American Society for Transplantation and Cellular Therapy; CL = clearance; CR = complete response; CRS = cytokine-release syndrome; CTCAE = Common Terminology Criteria for Adverse Events; DOR = duration of response; EFS = event free survival; ICANS = immune effector cell-associated neurotoxicity syndrome; NALT = new anti-lymphoma treatment; NCI = National Cancer Institute; ORR = overall response rate; OS = overall survival; PFS = progression free survival; PR = partial response.

### Objectives and Endpoints for Both Dose Escalation and Dose Expansion

Secondary Objectives

| Pharmacokinetic Objectives | Corresponding Endpoints |
|---|---|
| To characterize the PK profile of mosunetuzumab in | For Mosunetuzumab SC: |
| combination with Compound A-S | Cₘₐₓ |
| To characterize the PK profile of glofitamab in | Cₘᵢₙ |
| combination with Compound A-S | Total exposure (AUC), apparent CL, and apparent |
| To characterize the PK of Compound A-S when | volume of distribution, as estimated by population PK |
| administered in combination with mosunetuzumab | modeling, as appropriate, and supported by data |
| in Arm 1 and in combination with glofitamab in | For Glofitamab IV: |
| Arm 2 | Cₘₐₓ |
| | Cₘᵢₙ |
| | Total exposure (AUC), CL, and volume of distribution, |
| | as estimated by population PK modeling, as |
| | appropriate, and supported by data |
| | For Compound A-S: |
| | Cₘₐₓ |
| | Cₘᵢₙ |
| | Total exposure (AUC), CL, and volume of distribution, |
| | as estimated by population PK modeling, as |
| | appropriate, and supported by data |

Exploratory Objectives

| | Corresponding Endpoints |
|---|---|
| Pharmacokinetic Objectives | |
| To assess potential PK interactions between | PK parameters for mosunetuzumab given in |
| mosunetuzumab and Compound A-S | combination with Compound A-S compared with |
| To assess potential PK interactions between | mosunetuzumab given alone based on historical data |
| glofitamab and Compound A-S | PK parameters for Compound A-S given in |
| | combination with mosunetuzumab compared with |
| | Compound A-S given alone based on historical data |
| | PK parameters for Compound A-S given in |
| | combination with glofitamab compared with |
| | Compound A-S given alone based on historical data |
| To evaluate potential relationships between drug | Relationship between PK and safety, biomarker, or |
| exposure and efficacy, biomarker, and safety | efficacy endpoints, as appropriate |
| Immunogenicity Objectives | |
| To evaluate potential effects of ADAs of | For Arm 1: |
| mosunetuzumab when administered in combination | Relationship between ADA status and efficacy, safety, |
| with Compound A-S in Arm 1 | or PK endpoints |
| To evaluate potential effects of ADAs of glofitamab | For Arm 2: |
| when administered in combination with Compound | Relationship between ADA status and efficacy, safety, |
| A-S in Arm 2 | or PK endpoints |
| Biomarker Objectives | |
| To identify and/or evaluate biomarkers that are | Association between prognostic subtypes, exploratory |
| predictive of response (i.e., predictive biomarkers), | biomarkers, and safety and efficacy endpoints |
| are early surrogates of efficacy, are associated with | Relationship between biomarkers in blood and tumor |
| progression to a more severe disease state | tissue, like immune cells (e.g., T-cell |
| (i.e., prognostic biomarkers), are associated with | activation/exhaustion/reinvigoration) and inflammatory |
| acquired resistance, are associated with | cytokine release, and efficacy, safety, PK, |
| susceptibility to developing adverse events or can | immunogenicity, or other biomarker endpoints |
| lead to improved adverse event monitoring or | Tumor mutations and transcriptomic markers |
| investigation (i.e., safety biomarkers), can provide | (e.g., mutational status, gene expression), association |
| evidence of activity (i.e., pharmacodynamic | between MRD and efficacy endpoints, as well as |
| biomarkers), or can increase the knowledge and | safety, PK, or other biomarkers |
| understanding of disease biology in participants | Additional biomarkers may be explored depending on |
| treated with mosunetuzumab + Compound A-S, or | emerging data |
| glofitamab + Compound A-S | |

ADA = anti-drug antibody; AUC = area under the concentration-time curve; CL = clearance; Cₘₐₓ = maximum serum concentration observed; Cₘᵢₙ = minimum serum concentration under steady-state conditions within a dosing interval; MRD = minimal residual disease; PK = pharmacokinetic.

### Overall Design

This study is a Phase 1 b, open-label, multi-center study to evaluate the safety, efficacy, PK and PD of Compound A-S in combination with glofitamab via the intravenous route of administration or mosunetuzumab via the subcutaneous route of administration in participants with B-cell non-Hodgkin lymphoma, specifically participants with DLBCL, transformed FL (trFL), or FL (grades 1-3a).

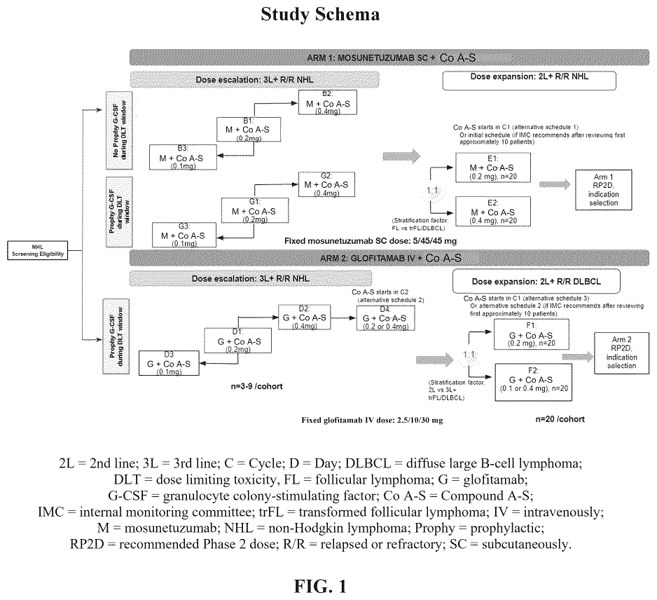

FIG. 1 provides a study schema. The following are the treatment combinations in participants with R/R NHL:
Arm 1: SC mosunetuzumab+Compound A-S, cohorts B1, B2, B3, G1, G2, G3 (dose escalation)
Arm 1: SC mosunetuzumab+Compound A-S, cohorts E1 and E2 (dose expansion)
Arm 2: IV glofitamab+Compound A-S, cohorts D1, D2, D3, D4 (dose escalation)
Arm 2: IV glofitamab+Compound A-S, cohorts F1 and F2 (dose expansion)

All participants will receive a total of 12 cycles with a cycle duration of 21 or 28 days. Some participants will receive prophylactic and/or therapeutic granulocyte colony-stimulating factor (G-CSF).

Arm 1: Mosunetuzumab Subcutaneously in Combination with Compound A-S Oral

PART 1: Under the initial dosing schedule, a fixed step-up dosing of mosunetuzumab (5/45/45 mg on Day 1, 8 and 15 of Cycle 1 respectively [21-day cycle]; 45 mg on Day 1 of Cycles 2-12 [28-day cycles]) will be administered SC concurrently with Compound A-S starting in Cycle 2 (FIG. 2). About two dose levels of Compound A-S will be tested in the dose escalation phase. Approximately 3-9 participants with B-cell NHL will be enrolled in each cohort.

PART 2: In the dose expansion phase, participants with at least one prior line of systemic lymphoma therapy will be randomized in a 1:1 ratio to receive either 0.2 mg/d or 0.4 mg/d of Compound A-S, where mosunetuzumab is administered in combination with Compound A-S under alternative dosing schedule 1 (Compound A-S starts in Cycle 1) to assess the impact of initiating Compound A-S during an earlier cycle. Under alternative dosing schedule 1, a fixed step-up dosing of mosunetuzumab (5/45/45 mg on Day 1, 8 and 15 of Cycle 1 respectively [28-day cycle]; 45 mg on Day 1 of Cycles 2-12 [28-day cycles]) will be administered SC concurrently with Compound A-S starting in Cycle 1 (FIG. 3).

Arm 2: Glofitamab Intravenously in Combination with Compound A-S Oral

PART 1: Under the initial dosing schedule, prior to glofitamab IV administration, participants will receive pre-treatment with a fixed dose of 1,000 mg obinutuzumab on Day 1 of Cycle 1. A fixed step-up dosing of glofitamab (2.5/10 mg on Day 8 and 15 of Cycle 1 respectively; 30 mg on Day 1 of Cycles 2-12) will be administered with Compound A-S starting in Cycle 3 (FIG. 4), 21 days per cycle. About two dose levels of Compound A-S will be tested in the dose escalation phase. After at least one dose level has cleared, the highest cleared dose level will be studied under alternative dosing schedule 2 (Compound A-S starts in Cycle 2) in Cohort D4 to assess the impact of initiating Compound A-S during an earlier cycle. Under alternative dosing schedule 2, a fixed step-up dosing of glofitamab (2.5/10 mg on Day 8 and 15 of Cycle 1 respectively; 30 mg on Day 1 of Cycles 2-12) will be administered with Compound A-S starting in Cycle 2 (FIG. 5), 21 days per cycle. Approximately 3-9 participants with B-cell NHL will be enrolled in each cohort.

PART 2: In the dose expansion phase, participants will be randomized in a 1:1 ratio to receive one of two selected dose levels of Compound A-S, where glofitamab is administered in combination with Compound A-S. If a dose level administered according to alternative dosing schedule 2 is cleared during dose escalation, participants with at least one prior line of systemic lymphoma therapy will receive Compound A-S under alternative dosing schedule 3 (Compound A-S starts in Cycle 1) during dose expansion to assess the impact of initiating Compound A-S during an earlier cycle. Under alternative dosing schedule 3, a fixed step-up dosing of glofitamab (2.5/10 mg on Day 8 and 15 of Cycle 1 respectively; 30 mg on Day 1 of Cycles 2-12) will be administered with Compound A-S starting in Cycle 1 (FIG. 6), 21 days per cycle.

### Study Population

Approximately 119-125 participants with B-cell NHL will be enrolled in this study (approximately 68 participants in Arm 1 and 51-57 participants in Arm 2).

### Inclusion Criteria

Participants must meet the following inclusion criteria to be eligible for enrollment into the study:
Age ≥18 years
Eastern Cooperative Oncology Group (ECOG) Performance Status of 0, 1, or 2
History of one of the following histologically documented hematologic malignancies that are expected to express the CD20 antigen: In the Dose Escalation phase, patients with R/R NHL who previously received at least two prior lines of systemic therapies can be enrolled. In the Dose Expansion phase, patients with FL (grade 1-3a), DLBCL/transformed FL who failed to respond to at least one prior line of systemic therapy can be potentially enrolled
Fluorodeoxyglucose-avid lymphoma (i.e. PET-positive lymphoma)
At least one bi-dimensionally measurable nodal lesion (>1.5 cm in its largest dimension by diagnostic quality CT or PET/CT scan), or at least one bi-dimensionally measurable extranodal lesion (>1.0 cm in its largest dimension by diagnostic quality CT or PET/CT scan)
Availability of a representative tumor specimen and the corresponding pathology report for confirmation of the diagnosis of NHL
A fresh pre-treatment biopsy during screening period, excisional or incisional, is preferred
Adequate hematologic function without growth factors or blood product transfusion within 14 days of first dose of study drug administration
Normal laboratory values
All participants and health care providers will be trained and counseled on pregnancy prevention. For biologically female participants of childbearing potential: agreement to remain abstinent (refrain from heterosexual intercourse) or use contraception during the treatment period and for 3 months after the final dose of mosunetuzumab, at least 18 months after pre-treatment with obinutuzumab or 2 months after the last dose of glofitamab, and 28 days after the last dose of Compound A-S, whichever is longer
For biologically male participants: agreement to remain abstinent (refrain from heterosexual intercourse) or use a condom, and agree to refrain from donating sperm during the treatment period and for at least 3 months after pre-treatment with obinutuzumab or 2 months after the last dose of glofitamab, and 28 days after the last dose of Compound A-S, whichever is longer

### Exclusion Criteria

Participants with any of the following characteristics/conditions are excluded:
Pregnancy or breastfeeding, or intention of becoming pregnant during the study (biologically female participants of childbearing potential must have a negative serum pregnancy test result within 14 days prior to initiation of the study treatment)
Participant has received prior therapy with cereblon (CRBN)-modulating drug (e.g., lenalidomide, avadomide/CC-122, pomalidomide) 4 weeks prior to starting Compound A-S
Inability to swallow pills, or persistent diarrhea or malabsorption ≥Grade 2 National Cancer Institute (NCI) Common Terminology Criteria for Adverse Events (CTCAE), despite medical management
QTc interval of >470 ms
The following treatments prior to study entry: mosunetuzumab, glofitamab, or other CD20/CD3-directed bispecific antibodies; allogenic stem cell therapy (SCT); solid organ transplantation
Treatments (investigation or approved) within the following time periods prior to initiation/first dose of study treatment: radiotherapy within 2 weeks; autologous SCT within 100 days; chimeric antigen receptor (CAR) T-cell therapy within 30 days; prior anti-lymphoma treatment with monoclonal antibodies or antibody-drug conjugates within 4 weeks; use of radioimmunoconjugates within 12 weeks; systemic immunosuppressive medications within 2 weeks; any other anti-cancer therapy, whether investigational or approved, including but not limited to chemotherapy, within 4 weeks or 5 half-lives of the drug, whichever is shorter
Live, attenuated vaccine within 4 weeks before first dose of study treatment, or in whom it is anticipated that such a live attenuated vaccine will be required during the study period or within 5 months after the final dose of study treatment
Current or past history of central nervous system (CNS) lymphoma or leptomeningeal infiltration
History of severe allergic or anaphylactic reactions to humanized or murine monoclonal antibody therapy (or recombinant antibody-related fusion proteins)
History of autoimmune disease, including but not limited to myocarditis, pneumonitis, myasthenia gravis, myositis, autoimmune hepatitis, systemic lupus erythematosus, rheumatoid arthritis, inflammatory bowel disease, vascular thrombosis associated with antiphospholipid syndrome, granulomatosis with polyangiitis, Sjögren's syndrome, Guillain-Barre syndrome, multiple sclerosis, vasculitis, or glomerulonephritis
Major surgery or significant traumatic injury <28 days prior to enrollment (excluding biopsies) or anticipation of the need for major surgery during study treatment
Clinically significant toxicities from prior treatment have not resolved to Grade 1 (per US national cancer institute (NCI) common terminology criteria for adverse events (CTCAE) v5.0) prior to the first study drug administration with exceptions defined by the protocol
Evidence of any significant, concomitant disease (e.g. cardiovascular, pulmonary, liver, CVA or stroke, ILD, PML, infection, HLH, etc.) that could affect compliance with the protocol or interpretation of results
For participants enrolled into glofitamab cohort: documented refractoriness to an obinutuzumab monotherapy-containing regimen (defined as disease that did not achieve response (PR or CR) or progressed within 6 months of the last dose of an obinutuzumab-containing regimen)

### Study Arms and Duration

### Treatment Period

In Arm 1 dose escalation (PART 1), participants receive mosunetuzumab subcutaneously (SC) in combination with Compound A-S by mouth (PO) in eleven 28-day cycles (plus initial 21-day priming dose cycle for mosunetuzumab) as detailed below (initial schedule):
Cycle 1 Day 1: Mosunetuzumab SC 5 mg
Cycle 1 Day 8: Mosunetuzumab SC 45 mg
Cycle 1 Day 15: Mosunetuzumab SC 45 mg
Cycle 2 to Cycle 12 Day 1: Mosunetuzumab SC 45 mg
Cycle 2 to Cycle 12 Days 1-14: Compound A-S PO at the specified dose (0.1 mg or 0.2 mg or 0.4 mg depending on the cohort assigned)

In Arm 1 dose expansion (PART 2), participants receive mosunetuzumab subcutaneously (SC) in combination with Compound A-S by mouth (PO) in twelve 28-day cycles as detailed below (alternative schedule 1):
Cycle 1 Day 1: Mosunetuzumab SC 5 mg
Cycle 1 Day 8: Mosunetuzumab SC 45 mg
Cycle 1 Day 15: Mosunetuzumab SC 45 mg
Cycle 1 Days 1-14: Compound A-S PO at the specified dose (0.1 mg or 0.2 mg or 0.4 mg depending on the cohort assigned)
Cycle 2 to Cycle 12 Day 1: Mosunetuzumab SC 45 mg
Cycle 2 to Cycle 12 Days 1-14: Compound A-S PO at the specified dose (0.1 mg or 0.2 mg or 0.4 mg depending on the cohort assigned)

In Arm 2 dose escalation (PART 1), participants receive glofitamab intravenously (IV) in combination with Compound A-S by mouth (PO) in ten 21-day cycles (plus two initial 21-day priming dose cycles for glofitamab) as detailed below (initial schedule):
Cycle 1 Day 1: Obinutuzumab IV 1,000 mg
Cycle 1 Day 8: Glofitamab IV 2.5 mg
Cycle 1 Day 15: Glofitamab IV 10 mg
Cycle 2 Day 1: Glofitamab IV 30 mg
Cycle 3 to Cycle 12 Day 1: Glofitamab IV 30 mg
Cycle 3 to Cycle 12 Days 1-10: Compound A-S PO at the specified dose (0.1 mg or 0.2 mg or 0.4 mg depending on the cohort assigned)

In Arm 2 dose escalation (PART 1), participants receive glofitamab intravenously (IV) in combination with Compound A-S by mouth (PO) in eleven 21-day cycles (plus initial 21-day priming dose cycle for glofitamab) as detailed below (alternative schedule 2):
Cycle 1 Day 1: Obinutuzumab IV 1,000 mug
Cycle 1 Day 8: Glofitamab IV 2.5 mg
Cycle 1 Day 15: Glofitamab IV 10 mug
Cycle 2 to Cycle 12 Day 1: Glofitamab IV 30 mg
Cycle 2 to Cycle 12 Days 1-10: Compound A-S PO at the specified dose (0.1 mg or 0.2 mg or 0.4 mg depending on the cohort assigned)

In Arm 2 dose expansion (PART 2), participants receive glofitamab intravenously (IV) in combination with Compound A-S by mouth (PO) in twelve 21-day cycles as detailed below (alternative schedule 3):
Cycle 1 Day 1: Obinutuzumab IV 1,000 mg
Cycle 1 Day 8: Glofitamab IV 2.5 mg
Cycle 1 Day 15: Glofitamab IV 10 mg
Cycle 1 Days 1-10: Compound A-S PO at the specified dose (0.1 mg or 0.2 mg or 0.4 mg depending on the cohort assigned)
Cycle 2 to Cycle 12 Day 1: Glofitamab IV 30 mg
Cycle 2 to Cycle 12 Days 1-10: Compound A-S PO at the specified dose (0.1 mg or 0.2 mg or 0.4 mg depending on the cohort assigned)

For each schedule, participants receive study interventions until disease progression, unacceptable toxicity, withdrawal of consent, or study termination. The total study duration for each participant is at least 2 years because all participants are followed for 2 years following last dose of treatment or until progressive disease and/or the start of new anti-lymphoma treatment, whichever is earlier.

### Example 2: Interim Results from a Phase 1b, Open-Label Study of Compound A-S in Combination with Glofitamab or Mosunetuzumab in Participants with B-Cell Non-Hodgkin Lymphoma

### Preliminary Safety and Efficacy Analysis

Interim results from Example 1 are presented.

Methods: Patients with R/R BCL (DLBCL, FL Grade 1-3a, trFL) received treatment for up to 12 cycles. In Arm 1, subcutaneous mosunetuzumab was given with step-up dosing in Cycle (C)1 (Day [D]1, 5 mg; D8 and D15, 45 mg; 21-day cycle), then 45 mg on D1 of subsequent cycles (28-day cycles); concurrently, oral compound A-S(0.1, 0.2, or 0.4 mg) was given daily from C1 or C2 onwards (D1-14; 28-day cycles). In Arm 2, patients received intravenous obinutuzumab pretreatment on C1D1 and intravenous glofitamab on C1D8 (2.5 mg), D15 (10 mg), then D1 of subsequent cycles (30 mg); oral compound A-S was given daily from C2 or C3 onwards (D1-10; 21-day cycles) in dose escalation (0.2 or 0.4 mg). Prophylactic G-CSF was mandatory for the first cycle of combination therapy. Primary endpoints were safety including dose-limiting toxicities (DLTs) and Compound A-S oral dose selection. Secondary endpoints included investigator-assessed overall response rate (ORR) and complete response rate (CRR; Lugano 2014 criteria). As of May 5, 2025, 35 patients (10 DLBCL; 20 FL; 4 trFL; 1 composite FL/DLBCL) and 12 patients (3 DLBCL; 9 FL) were enrolled in Arm 1 (subcutaneously administered mosunetuzumab+Compound A-S) and Arm 2 (intravenously administered glofitamab+Compound A-S), respectively (safety-evaluable population). In Arms 1 and 2, the median age was 63 years (range: 30-83) and 60 years (range: 37-76), 29% and 42% of patients had prior CAR T-cell therapy, respectively. Median number of prior therapies was 3 (range: 1-6) in Arm 1 and 2 (range: 1-4) in Arm 2. Grade ≥3 adverse events occurred in 74% and 67% of patients in Arms 1 and 2, respectively; there were no grade 5 adverse events. Serious adverse events occurred in 66% of patients in Arm 1 and 50% of patients in Arm 2. Most common adverse events in Arms 1 and 2, respectively, were neutropenia (grade ≥3: 57% and 58%), injection site reactions (57%; all grade ½ in Arm 1 only), cytokine-release syndrome (CRS) (grade ½: 43% and 33%, no grade ≥3), rash (grade ≥3: 3% and 0%), and infections (grade ≥3: 11% and 8%). Grade ≥3 neurological adverse events occurred in 3% of patients (n=1; grade 3 presyncope) in Arm 1 and 17% of patients (n=2; grade 3 cognitive disorder and grade 4 spinal cord compression) in Arm 2. ICANS as a preferred term was reported in one patient in Arm 2 (grade 2); suspected ICANS was observed in 11% of patients (all grade 1: cognitive disorder, n=1; lethargy, n=1; syncope, n=2) in Arm 1 and 17% of patients (grade 3 cognitive disorder, n=1; grade ½ somnolence, n=1) in Arm 2. Febrile neutropenia (FN) occurred in 11% of patients in Arm 1 and 8% of patients in Arm 2. Adverse events leading to treatment discontinuation occurred in 17% of patients (neutropenia, n=3; anemia, disseminated intravascular coagulation, and thrombocytopenia, n=1 each) in Arm 1 and none in Arm 2. Dose-limiting toxicities (DLTs) were reported in 3 patients in Arm 1 (Compound A-S 0.2 mg, grade 3 FN and herpes zoster; Compound A-S 0.1 mg, grade 3 rash) and one patient in Arm 2 (Compound A-S 0.4 mg, grade 3 FN).

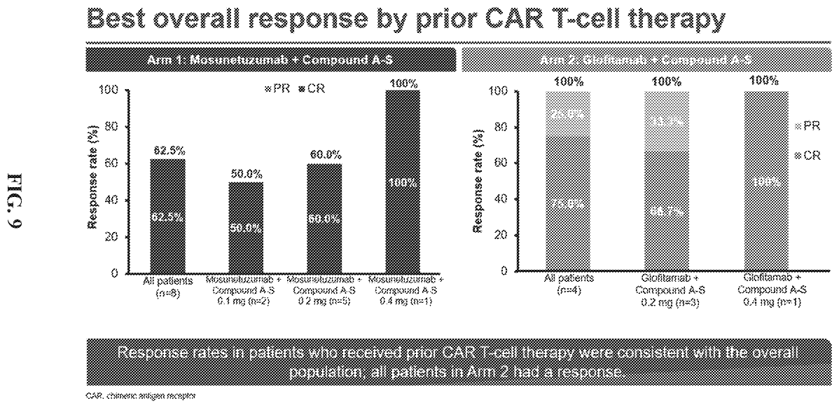

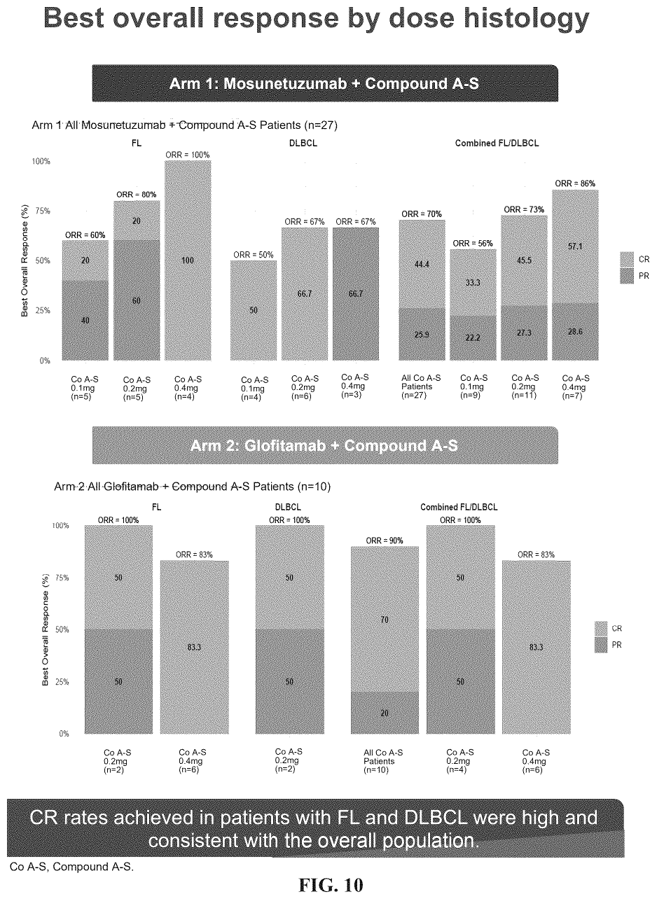

In efficacy-evaluable patients (Arm 1, n=27 [14 FL; 13 DLBCL/trFL]; Arm 2, n=10 [8 FL; 2 DLBCL/trFL]), responses were observed at all doses of Compound A-S in both arms. In Arm 1, the overall response rate (ORR) and the complete response rate (CRR) were 70% and 44%, respectively. In patients with FL, the ORR and CRR were 79% and 43%, and in DLBCL/trFL, 62% and 46%, respectively. At the 0.4 mg dose of Compound A-S(n=7), 4/4 patients with FL had a complete response (CR), and ⅔ patients with DLBCL/trFL had a partial response (PR). In Arm 2, the ORR and CRR were 90% and 70%, respectively. In 8 patients with FL, the ORR was 88% (⅞), CRR was 75% ( 6/8). Of the 2 patients with DLBCL/trFL, one had a CR, and one a PR. Median time to first response was 2.6 months (range: 2-4) in Arm 1 and 1.9 months (range: 1-3) in Arm 2. Ikaros degradation in peripheral T-cells was observed at 0.2 mg and 0.4 mg doses of Compound A-S. The preliminary results are also summarized in FIG. 8 to 10 showing respectively best overall response and DOR, best overall response by prior CAR T-cell therapy, and best overall response by dose histology.

### CONCLUSIONS

The above data show that Compound A-S in combination with mosunetuzumab or glofitamab is an active regimen with a manageable safety profile. No new safety signals were observed; CRS events were low grade. Neutropenia was the most common overlapping toxicity and was manageable with prophylactic and therapeutic G-CSF. ICANS as a preferred term was low grade and occurred in one patient. This novel combination therapy showed activity in heavily pretreated patients with B-cell non-Hodgkin lymphoma.

The examples set forth above are provided to give those of ordinary skill in the art a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1.-127. (canceled)

128. A method of treating or managing non-Hodgkin lymphoma (NHL), comprising administering to a subject having the NHL:
(a) a therapeutically effective amount of a compound, wherein the compound is Compound A 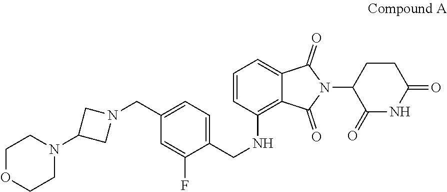 [SMILES: O=C1CCC(N2C(=O)c3cccc(NCc4ccc(CN5CC(N6CCOCC6)C5)cc4F)c3C2=O)C(=O)N1]
or an enantiomer or mixture of enantiomers, a tautomer, an isotopolog, a pharmaceutically acceptable salt, solvate, hydrate, co-crystal, clathrate, or polymorph thereof, in combination with
(b) a therapeutically effective amount of an anti-CD20/anti-CD3 bispecific antibody, wherein the bispecific antibody is glofitamab or mosunetuzumab.

129. The method of claim 128, wherein the compound is Compound A-S 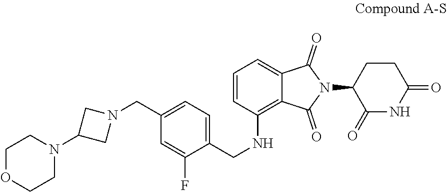 [SMILES: O=C1CC[C@H](N2C(=O)c3cccc(NCc4ccc(CN5CC(N6CCOCC6)C5)cc4F)c3C2=O)C(=O)N1]
or a tautomer, an isotopolog, a pharmaceutically acceptable salt, solvate, hydrate, co-crystal, clathrate, or polymorph thereof.

130. The method of claim 129, wherein the compound is administered in an amount of about 0.1 mg, about 0.2 mg, or about 0.4 mg per day.

131. The method of claim 130, wherein the bispecific antibody is glofitamab.

132. The method of claim 131, wherein the glofitamab is administered intravenously.

133. The method of claim 132, wherein administering the therapeutically effective amount of glofitamab comprises administering glofitamab according to a dosing regimen comprising at least a first dosing cycle (C1) and a second dosing cycle (C2), wherein:
(a) the first dosing cycle (C1) comprises a first dose (C1D1) and a second dose (C1D2) of glofitamab, wherein the C1D1 is about 2.5 mg, and the C1D2 is about 10 mg; and
(b) the second dosing cycle (C2) comprises a single dose (C2D1) of glofitamab, wherein the C2D1 is about 30 mg.

134. The method of claim 133, wherein the first dosing cycle (C1) further comprises a single dose (OD1) of obinutuzumab, wherein the OD1 is about 1,000 mg.

135. The method of claim 134, wherein the length of the first dosing cycle (C1) is about 21 days.

136. The method of claim 133, comprising administering to the subject the C1D1 and the C1D2 on or about days 8 and 15, respectively, of the first dosing cycle (C1).

137. The method of claim 133, wherein the first dosing cycle (C1) further comprises a single dose (OD1) of obinutuzumab, wherein the OD1 is about 1,000 mg, and wherein the dosing regimen comprises administering to the subject the OD1, the C1D1 and the C1D2 on or about days 1, 8 and 15, respectively, of the first dosing cycle (C1).

138. The method of claim 132, wherein administering the therapeutically effective amount of glofitamab comprises administering glofitamab intravenously according to a dosing regimen comprising a first dosing cycle (C1), a second dosing cycle (C2) and one to ten additional dosing cycles (C3-C12), wherein:
(a) the length of the first dosing cycle (C1) is about 21 days and the first dosing cycle (C1) comprises on day 1 a single dose (OD1) of obinutuzumab, on day 8 a first dose (C1D1) of glofitamab and on day 15 a second dose (C1D2) of glofitamab, wherein the OD1 is about 1,000 mg, the C1D1 is about 2.5 mg, and the C1D2 is about 10 mg;
(b) the length of the second dosing cycle (C2) is about 21 days and the second dosing cycle (C2) comprises on day 1 a single dose (C2D1) of glofitamab, wherein the C2D1 is about 30 mg; and
(c) the length of the one to ten additional dosing cycles (C3-C12) is about 21 days and the one to ten additional dosing cycles (C3-C12) comprise on day 1 a single dose (AD1) of glofitamab, wherein the AD1 is about 30 mg;
wherein the compound is administered orally in an amount of about 0.1 mg, about 0.2 mg or about 0.4 mg per day on days 1 to 10 of (i) the one to ten additional dosing cycles (C3-C12), or (ii) the second dosing cycle (C2) and the one to ten additional dosing cycles (C3-C12), or (iii) the first dosing cycle (C1), the second dosing cycle (C2) and the one to ten additional dosing cycles (C3-C12).

139. The method of claim 128, wherein the bispecific antibody is mosunetuzumab.

140. The method of claim 129, wherein the bispecific antibody is mosunetuzumab.

141. The method of claim 140, wherein the mosunetuzumab is administered subcutaneously.

142. The method of claim 139, wherein administering the therapeutically effective amount of mosunetuzumab comprises administering mosunetuzumab according to a dosing regimen comprising at least a first dosing cycle (C1) and a second dosing cycle (C2), wherein:
(a) the first dosing cycle (C1) comprises a first dose (C1D1), a second dose (C1D2), and a third dose (C1D3) of mosunetuzumab, wherein the C1D1 is about 5 mg, the C1D2 is about 45 mg, and the C1D3 is about 45 mg; and
(b) the second dosing cycle (C2) comprises a single dose (C2D1) of mosunetuzumab, wherein the C2D1 is about 45 mg.

143. The method of claim 142, wherein the compound is administered on days 1 to 14 in an amount of about 0.1 mg, about 0.2 mg or about 0.4 mg per day.

144. The method of claim 140, wherein administering the therapeutically effective amount of mosunetuzumab comprises administering mosunetuzumab according to a dosing regimen comprising at least a first dosing cycle (C1) and a second dosing cycle (C2), wherein:
(a) the first dosing cycle (C1) comprises a first dose (C1D1), a second dose (C1D2), and a third dose (C1D3) of mosunetuzumab, wherein the C1D1 is about 5 mg, the C1D2 is about 45 mg, and the C1D3 is about 45 mg; and
(b) the second dosing cycle (C2) comprises a single dose (C2D1) of mosunetuzumab, wherein the C2D1 is about 45 mg.

145. The method of claim 144, wherein the compound is administered on days 1 to 14 in an amount of about 0.1 mg, about 0.2 mg or about 0.4 mg per day.

146. The method of claim 141, wherein administering the therapeutically effective amount of mosunetuzumab comprises administering mosunetuzumab subcutaneously according to a dosing regimen comprising a first dosing cycle (C1) and one to eleven additional dosing cycles (C2-C12), wherein:
(a) the length of the first dosing cycle (C1) is about 21 days and the first dosing cycle (C1) comprises on day 1 a first dose (C1D1), on day 8 a second dose (C1D2), and on day 15 a third dose (C1D3) of mosunetuzumab, wherein the C1D1 is about 5 mg, the C1D2 is about 45 mg, and the C1D3 is about 45 mg; and
(b) the length of the one to eleven additional dosing cycles (C2-C12) is about 28 days and the one to eleven additional dosing cycles (C2-C12) comprise on day 1 a single dose (AD1) of mosunetuzumab, wherein the AD1 is about 45 mg;
wherein the compound is administered orally in an amount of about 0.1 mg, about 0.2 mg or about 0.4 mg per day on days 1 to 14 of the one to eleven additional dosing cycles (C2-C12).

147. The method of claim 141, wherein administering the therapeutically effective amount of mosunetuzumab comprises administering mosunetuzumab subcutaneously according to a dosing regimen comprising a first dosing cycle (C1) and one to eleven additional dosing cycles (C2-C12), wherein:
(a) the length of the first dosing cycle (C1) is about 28 days and the first dosing cycle (C1) comprises on day 1 a first dose (C1D1), on day 8 a second dose (C1D2), and on day 15 a third dose (C1D3) of mosunetuzumab, wherein the C1D1 is about 5 mg, the C1D2 is about 45 mg, and the C1D3 is about 45 mg; and
(b) the length of the one to eleven additional dosing cycles (C2-C12) is about 28 days and the one to eleven additional dosing cycles (C2-C12) comprise on day 1 a single dose (AD1) of mosunetuzumab, wherein the AD1 is about 45 mg;
wherein the compound is administered orally in an amount of about 0.1 mg, about 0.2 mg or about 0.4 mg per day on days 1 to 14 of the first dosing cycle (C1) and the one to eleven additional dosing cycles (C2-C12).
